(19) United States
(12) Patent Application Publication
(10) Pub. No.: US 2003/0044821 A1
(43) Pub. Date: Mar. 6, 2003

(54) DNA pooling methods for quantitative traits using unrelated populations or sib pairs

(76) Inventors: Joel S. Bader, Stamford, CT (US); Aruna Bansal, Branford, CT (US); Pak Sham, London (GB)

Correspondence Address: MINTZ, LEVIN, COHN, FERRIS,, GLOVSKY AND POPEO, P.C., One Financial Center, Boston, MA, 02111, US

(21) Appl. No.: 10/131,447

(22) Filed: Apr. 22, 2002

Related U.S. Application Data
(63) Continuation of application No. 09/932,480, filed on Aug. 17, 2001, now abandoned.
(60) Provisional application No. 60/226,465, filed on Aug. 18, 2000.
(60) Provisional application No. 60/230,580, filed on Sep. 5, 2000.

(51) Int. Cl. C12Q 1/68; G06F 19/00; G01N 33/48; G01N 33/50

(52) U.S. Cl. 435/6; 702/20

(57) ABSTRACT

Identifying the genetic determinants for disease and disease predisposition remains one of the outstanding goals of the human genome project. When large patient populations are available, genetic approaches using single nucleotide polymorphism markers have the potential to identify relevant genes directly. While individual genotyping is the most powerful method for establishing association, determining allele frequencies in DNA pooled on the basis of phenotypic value can also reveal association at a much-reduced cost. Here we analyze pooling methods to establish association between a genetic polymorphism and a quantitative phenotype. Exact results are provided for the statistical power for a number of pooling designs where the phenotype is described by a variance components model and the fraction of the population pooled is optimized to minimize the population requirements. For low to moderate sibling phenotypic correlation, unrelated populations are more powerful than sib pair populations with an equal number of individuals; for sibling phenotypic correlations above 75%, however, designs selecting the sib pairs with the greatest phenotype difference become more powerful. For sibling phenotype correlations below 75%, pooling extreme unrelated individuals is the most powerful design for sib pair populations. The optimal pooling fractions for each design are constant over a wide range of parameters. These results for quantitative phenotypes differ from those reported for qualitative phenotypes, for which unrelated populations are more powerful than sib pairs and concordant designs are more powerful than discordant, and have immediate relevance to ongoing association studies and anticipated whole-genome scans.

### RELATED APPLICATIONS

This application claims priority to U.S. Ser. No. 09/932,480, filed Aug. 17, 2001; U.S. Ser. No. 60/226,465 filed Aug. 18, 2000 [Cura 396], and to U.S. Ser. No. 60/230,580 filed Sep. 5, 2000 [Cura 396A], both of which are incorporated herein by reference in their entireties.

### BACKGROUND OF THE INVENTION

The complex diseases that present the greatest challenge to modem medicine, including cancer, cardiovascular disease, and metabolic disorders, arise through the interplay of numerous genetic and environmental factors. One of the primary goals of the human genome project is to assist in the risk-assessment, prevention, detection, and treatment of these complex disorders by identifying the genetic components. Disentangling the genetic and environmental factors requires carefully designed studies. One approach is to study highly homogenous populations (Nillson and Rose 1999; Rabinow, 1999; Frank 2000). A recognized drawback of this approach, however, is that disease-associated markers or causative alleles found in an isolated population might not be relevant for a larger population. An attractive alternative is to use well-matched case-control studies of a more diverse population. A second alternative is to study siblings, inherently matched for environmental effects.

Even with a well-matched sample set, the genetic factors contributing to an aberrant phenotype may be difficult to determine. Traditional linkage analysis methods identify physical regions of DNA whose inheritance pattern correlates with the inheritance of a particular trait (Liu 1997; Sham 1997, Ott 1999). These regions may contain millions of nucleotides and tens to hundreds of genes, and identifying the causative mutation or a tightly linked marker is still a challenge. A more recent approach is to use a sufficiently dense marker set to identify causative changes directly. Single nucleotide polymorphisms, or SNPs, can provide such a marker set (Cargill et al. 1999). These are typically bi-allelic markers with linkage disequilibrium extending an estimated 10,000 to 100,000 nucleotides in heterogeneous human populations (Kruglyak 1999; Collins et al. 2000). Tens to hundreds of thousands of these closely spaced markers are required for a complete scan of the 3 billion nucleotides in the human genome. Because each SNP constitutes a separate test, the significance threshold must be adjusted for multiple hypotheses (p-value˜10⁻⁸) to identify statistically meaningful associations. Consequently, hundreds to thousands of individuals are required for association studies (Risch and Merikangas 1996).

The most powerful tests of association require that each individual be genotyped for every marker (Fulker et al. 1995, Kruglyak and Lander 1995, Abecasis et al. 2000, Cardon 2000) and remain far too costly for all but testing candidate genes. An alternative that circumvents the need for individual genotypes, related to previous DNA pooling methods for determination of linkage between a molecular marker and a quantitative trait locus (Darvasi and Soller 1994), is to determine allele frequencies for sub-populations pooled on the basis of a qualitative phenotype. Populations of unrelated individuals, separated into affected and unaffected pools, have greater power than related populations. If a population consists of sib-pairs, concordant pairs versus unrelated controls have greater power than discordant pairs separated into affected and unaffected pools (Risch and Teng 1998). Nevertheless, discordant designs might provide a better control for confounding factors such as age, ethnicity, or environmental effects.

The phenotypes relevant for complex disease are often quantitative, however, and converting a quantitative score to a qualitative classification represents a loss of information that can reduce the power of an association study. The location of the dividing line for affected versus unaffected classification, for example, can affect the power to detect association. Furthermore, pooling designs based on a comparison of numerical scores are not even possible with a qualitative classification scheme. These distinctions can be especially relevant when populations contain related individuals and qualitative tests have a disadvantage (Risch and Teng 1998).

There remains a need for procedures that provide phenotype associations with diseases or pathologies based on phenotypes that may be ranked on a quantitative scale. In such a scheme there is a strong need to identify procedures for optimally obtaining samples, or pooling, from a subpopulation that provide the highest assurance of displaying associations that are present. In addition there is a need to distinguish among various pooling strategies that may arise in cases with different allele frequencies and different allele correlations. There is a further need to devise a test criterion for establishing the significance of associations between phenotypes and diseases or pathologies that may arise. The present invention addresses these and related deficiencies that currently exist.

### SUMMARY OF THE INVENTION

The present invention is based, in part, on the discovery of methods to detect an association in a population of individuals between a genetic locus and a quantitative phenotype, where two or more alleles occur at a given genetic locus, and the phenotype is expressed using a numerical phenotypic value whose range falls within a first numerical limit and a second numerical limit. These limits are used to provide for subpopulations that consist of upper and lower pools.

In some embodiments, the population of individuals includes individuals who may be classified into classes. In certain aspects of the invention, these classes are based on age, gender, race, or ethnic origin. In other aspects, some or all members of a class are included in the pools.

In various embodiments, these numerical limits are chosen so that the upper pool includes the highest 10%, 15%, 20%, 25%, 27%, 30%, or 35% of the population. In other embodiments, the numerical limits are chosen such that the lower pool includes the lowest 10%, 15%, 20%, 25%, 27%, 30%, or 35% of the population.

In one embodiment of the invention, the numerical limits are chosen to minimize false-negative errors.

In the present invention, the population of individuals can include unrelated individuals or related individuals. In one aspect, these related individuals are sibling pairs (sib pairs). In a further aspect, each member of the sib pair is selected for the upper pool. In a still further aspect, each member of the sib pair is selected for the lower pool. In still yet another aspect, neither member of the sib pair is selected. In another aspect, one member of the sib pair is selected for the upper pool and the other member of the sib pair is selected for the lower pool.

In one embodiment of the invention, sib pairs are ranked by the absolute magnitude of the difference in phenotypic value for the siblings within each pair. In one aspect, the percent of pairs with the greatest difference are identified, and the siblings in each pair are distributed such that the sibling with the high phenotypic value is selected for the upper pool and the sibling with the low phenotypic value is selected for the lower pool. In an aspect of this embodiment, the phenotypic value of one member of the sibling pair is above a predetermined lower limit and the phenotypic value of the second member of the sibling pair is below a predetermined upper limit. In various other aspects, the percentage of pairs with the greatest difference is 80%, 70%, 60%, 54% or 50%, and the distribution provides 10%, 15%, 20%, 25%, or 27% of the population in each pool.

In an embodiment of the invention, Mahalanobis ranks are generated among sib pairs. In one aspect, these ranks are used to construct pools composed of the member of the sib pair with the more extreme Mahalanobis rank. In another aspect, the Mahalanobis ranks are used to generate a list in which the order of each member of a sib pair in this list is determined by the smaller of the distance of a member from the first member on the list and the distance of a member from the last member on the list.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1. Shaded regions illustrate which siblings are selected under different pooling designs. The x-axis represents X₁, the phenotypic value for the first sibling, and the y-axis represents X₂, the value for the second sibling. The indicator functions I_{U1}, I_{U2}, I_{L1}, and I_{L2} take the value 1 when a sibling is selected for the denoted pool and are 0 otherwise. The unrelated-random design assumes a population of unrelated individuals, and only the first sibling is used. The pair-mean design depends on the sibling phenotype mean X₊=(X₁+X₂)/2; the pair-difference design depends on the difference X₋=(X₁−X₂)/2.

FIG. 2. The population A necessary to detect association is shown as a function of the pooling fraction ρ for three values of the sibling phenotype correlation r. Panel A: r=0.1, low correlation; Panel B: r=0.5, moderate correlation; Panel C: r=0.9, high correlation. The values of the remaining parameters are α5×10⁻⁸, 1−β=0.8,p₁=0.1, σ_{A}²=0.02, and d/a=0. For low to moderate sibling correlation, the unrelated-random design is more powerful than any design using sib pairs; for high sibling correlation, sib-apart designs are more powerful. The flat minima indicate that pooling fractions close to the minima are near optimal.

FIG. 3. The population N necessary to detect association is shown as a function of the sibling phenotype correlation r. The pooling fraction ρ is optimized to minimize the population requirements at specified false-positive rate α=5×10⁻⁸ and power 1−β=0.8 with remaining parameters p₁=0.1, σ_{A}²=0.02, and d/a=0. Panel A: Below r=0.75, the unrelated-random design is most powerful, followed by unrelated-extreme for sib pairs; above r=0.75, the pair-difference design is most powerful. The sib-apart designs are more powerful than sib-together designs above r=0.5 but are less powerful below this value. Panel B: The optimal pooling fraction is approximately 0.27 for the unrelated-random, pair-mean, pair-difference, and concordant designs; 0.18 for the unrelated-extreme design; and 0.23 for the discordant design. The optimal pooling fraction decreases for sib-apart designs in regions of large sibling correlation.

FIG. 4. The population N necessary to detect association is shown as a function of the minor-allele frequency p₁. The pooling fraction ρ is optimized to minimize the population requirements at specified false-positive rate α=5×10⁻⁸ and power 1−β=0.8 with remaining parameters r=0.4, α_{A}²=0.02, and d/a=0. Panel A: The population N is relative flat until p₁ falls below the additive variance Γ_{A}², at which point the phenotype becomes nearly monogenic and the population requirement decreases. Panel B: The optimal pooling fraction ρ is relative flat until p₁ falls below the additive variance σ_{A}², at which point it decreases rapidly.

FIG. 5. The population N necessary to detect association is shown as a function of the additive variance σ_{A}². The pooling fraction ρ is optimized to minimize the population requirements at specified false-positive rate α=5×10⁻⁸ and power 1−β=0.8 with remaining parameters r=0.4, p₁=0.1, and d/a=0. Panel A: The population requirement is inversely proportional to 1/σ_{A}², except for vary large values of σ_{A}² characteristic of a monogenic trait. Panel B: The optimal pooling fraction ρ is independent of σ_{A}² except for large values of σ_{A}².

FIG. 6. The population N necessary to detect association is shown for four values of the dominance ratio d/a as a function of the pooling fraction ρ. The remaining parameters are α=5×10⁻⁸, 1−β=0.8, r=0.4, p₁=0.1, and σ_{A}²=0.02. Panel A: d/a=−1 (pure recessive); Panel B: d/a=−0.9; Panel C: d/a=−0.5; Panel D: d/a=1 (pure dominant). These values were selected to sample the ratio of dominance variance to total variance for the allele, σ_{D}²/(σ_{D}²+σ_{A}²). Most association methods are more sensitive to additive variance than dominance variance. Close to d/a=1/(2p₁−1), the additive variance vanishes and the curve of N versus ρ changes from having a shallow minimum near ρ=0.27 (ρ=0.18 for unrelated-extreme) to being steeply sloped toward ρ=0. For rare alleles, this behavior occurs in a narrow region near d/a=−1 (pure recessive).

FIG. 7. The population N necessary to detect association is shown as a function of the dominance ratio d/a. Panel A: N when the pooling fraction ρ=0.2; Panel B: N when ρ has been optimized to minimize the population requirements for each value of d/a; Panel C: the optimized ρ. The remaining parameters are α=5×10⁻⁸, 1−β0.8, r=0.4, p₁=0.1, σ_{A}²=0.02. When ρ=0.2, near-optimal for alleles with additive variance, the population requirements increase markedly near d/a=−1 where the additive variance is small relative to the dominance variance for a low-frequency allele. The population requirements to detect rare recessive alleles could be reduced by decreasing ρ by 10-fold to 100-fold, but this would reduce the power to detect association for alleles outside of this narrow region of large dominance variance. The population requirements and the optimal pooling fraction are not sensitive to changes in d/a for low-frequency alleles that are under-dominant (d/a<−2), weakly recessive (d/a≈−0.5), additive (d/a=0), dominant (d/a=1), or over-dominent (d/a>1).

FIG. 8. The population N required to detect association is shown as a function of the Type I error rate α and the Type II error rate β. The pooling fraction ρ has been optimized to minimize the population size. Panel A: N is asymptotic to 2 ln(1/α) for small values of α. The remaining parameters are 1−β=0.8, r=0.4, p₁=0.1, σ_{A}²=0.02, and d/a=0. Panel B: The optimal pooling fraction ρ is not sensitive to changes in α. Panel C: The required population increases when β decreases. The remaining parameters are α=5×10⁻⁵, appropriate for a test of 1000 candidate polymorphisms versus a single phenotype, r=0.4,p₁=0.1, σ_{A}²=0.02, and d/a=0.

FIG. 9. The repository size required to detect association using pooled DNA is shown as a function of the fraction of population β selected for each pool, relative to the repository size required for a regression test using individual genotyping, for a QTL making a small contribution to a complex trait. The same family structure and the same phenotypic variable, either the individual phenotype, the pair-mean, the pair-difference, or the combined results from pair-mean and pair-difference tests, are used for tests based on pooling and individual genotyping. All of these tests show the same relative efficiency as a function of pooling fraction, with an optimal fraction of 0.27 requiring only 1.24× the population for individual genotyping. The Mahalanobis design is compared to the combined regression test for a sibling phenotypic correlation of t_{R}=0.6. The optimum occurs for this, and all other values of t_{R}, at ρ=0.188.

FIG. 10. The repository size required to detect association for the Mahalanobis design, relative to the population required for a combined regression test using individual genotypes, is shown as a function of the sibling phenotypic correlation t_{R}.

FIG. 11. The number of individuals required for pooling designs with a sib-pair family structure is compared to the number of unrelated individuals for an association test of equivalent power and significance as a function of the sibling phenotypic correlation t_{R}.

FIG. 12. (A) Exact numerical results for the repository size required to detect association are shown for pooling designs as a function of σ_{A}²/σ_{R}², the ratio of the additive variance of the QTL to the residual variance. The remaining parameters are allele frequency 0.1, additive inheritance, type I error 5×10⁻⁸, and type II error 0.2. (B) The allele frequency difference at significance is shown for the same parameters as in FIG. 12A. In this an all subsequent figures, unrelated-population is a dotted line, Mahalanobis a thin line, pair-mean a dashed line, pair-difference a dot-dashed line, and sib-combined a thick line.

FIG. 13. Exact numerical results for the repository size required to detect association is shown as a function of the allele frequency p for (A) dominant inheritance, (B) additive inheritance, and (C) recessive inheritance for tests using pooled DNA. The variance ratio σ_{A}²/σ_{R}² is 0.02, the type I error is 5×10⁻⁸, the type II error is 0.2, the pooling fraction 0.27 is used for all designs except Mahalanobis, for which 0.188 is used. The Mahalanobis design loses power for rare alleles faster than the other designs.

FIG. 14. Exact numerical results for the repository size required to detect association is shown as a function of the heterozygote phenotypic displacement d, describing the inheritance mode, for allele frequencies of (A)p=0.5, (B)p=0.25, and (C)p=0.1 for tests using pooled DNA. All other parameters are as in FIG. 13.

FIG. 15 The repository size required to detect association for a QTL for a complex trait is shown for pooled DNA designs relative to individual genotyping designs having equivalent type I and type II error rates. The ratio N_{aff/unaff}/N_{indiv} for affected/unaffected pools (dashed line) is shown as a function the disease prevalence r, while the ratio Nₜₐᵢₗ/N_{indiv} (solid line) is shown as a function of the fraction ρ of the total population selected for each pool. The optimum value of Nₜₐᵢₗ/N_{indiv} is 1.24 and occurs at σ=27.03% selected for each pool.

FIG. 16 The effect of varying the inheritance mode is shown for tail pools. The type I error is 5×10⁻⁸, the type II error rate is 0.2, and the displacement a is 0.25 in units of the phenotypic standard deviation. The displacement d of heterozygotes varies from −a, pure recessive inheritance, to +a, pure dominant inheritance. Three allele frequencies are shown, p=0.5, 0.1, and 0.01. Solid lines correspond to exact numerical calculations. (Top) The repository size N is shown. Filled circles corresponding to analytical approximations, Eq. 1, are virtually indistinguishable from exact calculations. (Bottom) The optimal pooling fraction ρ from numerical calculations falls in a narrow range from 24.5% to 27.5%, close to the analytical approximation of 27.03%.

FIG. 17 (Top) Exact numerical results for the repository size N required to achieve a type I error rate of 5×10⁻⁸ and type II error rate of 0.2 are shown for affected/unaffected pools (dashed line) and tail pools (solid line) as a function of the additive variance, also presented as the genotype relative risk for a heterozygote, for an allele with frequency 0.1 and purely additive inheritance. Analytical approximations (solid circles), Eqs. 1 and 2, are indistinguishable from the exact results when the genotype relative risk is smaller than 2. The disease prevalence r is 10% for the affected/unaffected pools, and 27% of the population is selected for each of the tail pools. (Bottom) The frequency difference at the significance threshold is shown for the same parameters. This threshold determines the measurement accuracy required for association tests based on pooled DNA.

### DETAILED DESCRIPTION OF THE INVENTION

1. Definitions

Glossary of Mathematical Symbols

| | |
|---|---|
| X | quantitative phenotypic value of an individual |
| Xᵢ | quantitative phenotypic value of sib i, with i = 1 or 2 for |
| | sib-pairs |
| X_{±} | (X₁ ± X₂)/2 |
| r | phenotypic correlation between sibs |
| Aᵢ | allele inherited at a particular locus. For a bi-allelic |
| | marker, i = 1 or 2 |
| G | genotype at the locus, either A₁A₁, A₁A₂, or A₂A₂ for a |
| | bi-allelic marker |
| Gᵢ | genotype for sib i, with i = 1 or 2 for sib-pairs |
| P(G) | genotype probability |
| P(G₁, G₂) | joint sib-pair genotype probability |
| f(X₁, X₂) | joint sib-pair phenotype probability distribution |
| f[X₁, X₂\|G₁, | joint sib-pair phenotype probability distribution |
| G₂] | conditioned on genotypes |
| p | frequency of allele A₁ in a population |
| q | frequency of the remaining alleles, with q = 1 − p |
| pᵢ | frequency of allele A₁ in sib i, either 1, 0.5, or 0 for an |
| | autosomal marker |
| p_{±} | (p₁ ± p₂)/2 |
| a | half the difference in the shift in the mean phenotypic |
| | value of individuals with genotype A₁A₁ compared to |
| | A₂A₂ |
| d | difference in the mean phenotypic value between |
| | individuals with genotype |
| A₁A₂ | compared to the mid-point of the means for A₁A₁ |
| | and A₂A₂ |
| μ | mean phenotypic shift due to the locus, equal to |
| | a(p − q) + 2pqd |
| σ_{A}² | additive variance of phenotype X due to the genotype G |
| σ_{D}² | dominance variance due to the genotype G |
| σ_{R}² | residual phenotypic variance, with σ_{A}² + σ_{D}² + σ_{R}² = 1 |
| N | the total number of individuals whose DNA is available |
| | for pooling |
| n | number of individuals selected for a single pool |
| ρ | pooling fraction defined as n/N |
| p_{U}, p_{L} | frequency of allele A₁ in the upper (U) or lower (L) pool |
| T | test statistic, which is expected to be close to zero when |
| | the genotype G does not affect the phenotypic value and |
| | is expected to be non-zero when individuals with |
| | genotypes A₁A₁, A₁A₂, and A₂A₂ have different mean |
| | phenotypic values. As formulated here, T has a normal |
| | distribution with unit variance. Under the null hypothesis |
| | that σ_{A} = (2pq)^{1/2}[α − (p − q)d] is zero, the mean of T |
| | is zero. Under the alternative hypothesis that σ_{A} is non- |
| | zero, the mean of T is also non-zero. |
| σ₀² | variance of n^{1/2}(p_{U} − p_{L}) under the null hypothesis |
| σ₁² | variance of n^{1/2}(p_{U} − p_{L}) under the alternative hypothesis |
| Φ(z) | cumulative standard normal probability, the area under a |
| | standard normal distribution up to normal deviate z |
| z_{α} | normal deviate corresponding to an upper tail area of α, |
| | defined as Φ(zₐ) = 1 − α |
| α | type I error rate (false-positive rate). For a one-sided test, |
| | T > zₐ corresponds to statistical significance at level α, |
| | typically termed a p-value. A typical threshold for |
| | significance is p-value smaller than 0.05 or 0.01. If M |
| | independent tests are conducted, a conservative |
| | correction that yields a final p-value of α is to |
| | use a p-value of α/M for each of the M tests. |
| β | type II error rate (false-negative rate). The power of a test |
| | is 1-β. |
| H(x) | Heaviside step function |

As used herein, when two individuals are “related to each other”, they are genetically related in a direct parent-child relationship or a sibling relationship. In a sibling relationship, the two individuals of the sibling pair have the same biological father and the same biological mother. As used herein, the term “sib” is used to designate the word “sibling”, and the sibling relationship is defined above. The term “sib pair” is used to designate a set of two siblings.

The members of a sib pair may be dizygotic, indicating that they originate from different fertilized ova. A sib pair includes dizygotic twins.

The focus of the present invention is to examine the statistical power of pooling designs for quantitative phenotypes. A variance components model provides the distribution of phenotypic values for an unselected population of unrelated individuals or sib pairs. The phenotype is partitioned into contributions from a specific causative allele and from residual shared and non-shared familial and genetic factors. The genotype-dependent phenotype distribution for sib pairs under Hardy-Weinberg equilibrium is used as the basis for analyzing the statistical power of various pooling strategies. The test statistic in each case is the allele frequency difference between two pools, appropriately standardized to a normal distribution. Numerically exact results are provided for a range of parameters including the fraction of population pooled, the allele frequency, and the dominant or recessive character of the allele. Furthermore, upon consideration of the relative powers of pooling designs, pooling designs are suggested for particular phenotype characteristics.

2. Model 1

2.1 Biometrical Genetic Model

A quantitative phenotype X, standardized to zero mean and unit variance, is hypothesized to be affected by the genotype G at a biallelic locus with alleles A₁ and A₂, occurring at population frequencies p₁ and p₂=1−p₁. More generally, A₂ may represent any of a number of alternate alleles, and p₂ their aggregate frequency. The population is assumed to be random mating, with genotype frequencies P(G) of p₁p₁, 2p₁p₂, and p₂p₂ for A₁A₁, A₁A₂, and A₂A₂ respectively. The frequency of allele p₁ in genotype G, denoted p_{G}, is 1 for A₁A₁, 0.5 for A₁A₂, and 0 for A₂A₂. The bivariate probability distribution P(G₁,G₂) of the 9 possible combinations of dizygotic sib-pair genotypes G₁ and G₂, shown in Table I, can be derived by considering all possible parental mating types and their offspring genotype distributions (Neale and Cardon 1992). The shared genetic makeup implies that P(G₁,G₂) ≈P(G₁)P(G₂).

Using the notation defined above, the effect μ_{G} of genotype G on the phenotype is a-μ. d-μ, and −a -μ for genotypes A₁A₁, A₁A₂, and A₂A₂ respectively. The constant μ=a(p₁−p₂)+2d p₁p₂ ensures that the phenotype has zero mean. The ratio d/a, termed the dominance ratio, is −11 for a recessive allele, +1 for a dominant allele, and 0 for an additive allele.

The phenotypic variance contributed by the genotype G can be partitioned into an additive component σ_{A}² and an dominance component σ_{D}², with

σ_{A}²2=2pq[a−d(p−q)]², and

σ_{D}²=4p²q²d².

In a population of unrelated individuals, the distribution f [X] of trait values is a mixture of 3 univariate normals, one for each genotype:

f[X]=Σ_{G}[X|μ_{G}]P(G), with

f[X|μ_{G}]=(2πσ_{R}²)^{-1/2}exp[−(X−μ_{G})²/2σ_{R}²]

and the residual variance σ_{R}²=1−σ_{A}²−σ_{D}².

Similarly, in a population of sib pairs, the bivariate distribution of trait values f [X₁X₂] is a mixture of 9 bivariate normals, appropriately weighted according to genotype combination:

$$f [ {X}_{1} , {X}_{2} ] = {\sum }_{{G}_{1} {G}_{2}} f [ {X}_{1} , {X}_{2} \vert {G}_{1} , {G}_{2} ] P [ {G}_{1} , {G}_{2} ] .$$

The mean of Xⱼ is μ_{G}_{₁} for sib j=1 or 2; both X₁ and X₂ have residual variance σ_{R}²=1−σ_{A}²−σ_{D}²; and X₁ and X₂ have correlation r_{R} due to shared residual polygenic effects and environmental factors. The total correlation r between sib pairs, including effects from genotype G, is

r=r_{R}+σ_{A}²/2+σ_{D}²/4

It is convenient to re-express the phenotypes of sib pairs in terms of X₊ and X₋, defined as the linear combinations X_{±}=(X₁±X₂)/2, because these components are uncorrelated and the probability distribution f[X₊,X₋|G₁,G₂] factors into the product f[X₊|G₁,G₂]f[X₋|G₁,G₂]. The individual probability distributions for X₊ and X₋ are

f[X_{±}|G₁,G₂]=(2πσ_{±}²)^{-1/2}exp[−(X_{±}−μ_{±})²/2σ_{±}²], with

μ_{±}(G₁,G₂)=(μ_{G}_{₁}±μ_{G}_{₂})/2 and

σ_{±}²=σ_{R}²(1+r_{R})/2.

Allele frequencies p_{±} are similarly defined as p_{±}(G₁,G₂)=(p_{G}_{₁}±p_{G}_{₂})/2.

2.2 Test Statistic and the Null Hypothesis

We consider tests in which an upper and lower pool, each containing n individuals, are selected according to higher and lower phenotypic values from a larger population of N individuals. The frequencies p_{U} and p_{L} of allele A₁A are calculated for the upper and lower pools, and the frequency difference is converted to the test statistic T,

T=(p_{U}−p_{L})/(σ₀/\sqrt{n}).

The variance p_{U}−p_{L} under the null hypothesis that genotype G has no effect on phenotype X is Var(p_{U}−p_{L})=σ₀²/n. When the null hypothesis is valid and n is large, T follows a standard normal distribution and σ₀ is independent of n. The value of σ₀ depends on the population allele frequencies and also on the method used to select the n individuals for each pool. Specifically, let n_{C} be the total number of sib pairs selected for the same pool and n_{D} be the number split between pools, with the remaining 2(n −n_{C}−n_{D} ) individuals unrelated. The contribution of the unrelated individuals to Var(p_{U}−p_{L}) is [2(n−n_{C}−n_{D})/n²]Var(p_{G}), and the individual variance is

Var(p_{G})=p₁²(1)+2p₁p₂(¼)−p₁²=p₁p₂/2.

The contribution of the pooled-together sib-pairs is

[n_{C}/n²]Var(p_{G}_{₁}+p_{G}_{₂})=[n_{C}/n²][2Var(p_{G})+2Cov(p_{G}_{₁}, p_{G}_{₂})]=(n_{C}/n²)(3p₁p₂/2)

because the covariance between genotypes in a sib-pair is half the individual variance, reflecting that sibs share half their genetic material. Similarly, the contribution of the pooled-apart sib-pairs is

[n_{D}/n²]Var(p_{G}_{₁}−p_{G}_{₂})=[n_{D}/n²][2Var(p_{G})−2Cov(p_{G}_{₁}, p_{G}_{₂})].

The result for σ₀² is

σ₀²=1+[n_{C}/2n)−(n_{D}/2n)]p₁p₂,

with important limiting cases of p₁p₂/2 for pure sib-apart pooling, p₁p₂ for pure unrelated pooling, and 3p₁p₂/2 for pure sib-together pooling. The allele frequency p may be determined from the entire population. It is also possible to estimates p₁ as the mean (p_{U}+p_{L})/2, which is closer to 0.5 than the population mean p₁ in the case of true association. The resulting σ₀ is larger, and using the mean results in a conservative test.

2.3 Pooling Design

A pooling design is a set of rules to determine which sibs are selected for the upper and lower pools. For an unrelated population, these rules take the form of a pair of indicator functions Iᵤ(X) for the upper pool and I_{L}(Y) for the lower pool. Each function takes the value 1 if an individual is selected for the specified pool and is 0 otherwise. In general, individuals are selected for at most one pool and Iᵤ+I_{L} is either 0 or 1.

The rules for sib-pairs may be formulated in terms of four indicator functions which depend on both sibling phenotypic values X₁ and X₂. These indicator functions may be written Iₛⱼ(X₁,X₂) or equivalently Iₛⱼ(X₊,X₋), where the side S is U or L and j=1 or 2 labels the sibling. The indicator function has value 1 if sib j is selected for side S and is 0 otherwise. As before, each individual is selected for at most one pool and I_{Uj} +I_{Lj} is either 0 or 1.

A summary of pooling designs in terms of the indicator functions is provided in Table II. The indicator functions are specified by upper and lower phenotype thresholds X_{U} and X_{L} and the Heaviside step function H(x),

$$H ( x ) = \{ \begin{matrix}1 , & x > 0 ; \\ 1 / 2 , & x = 0 ; \\ 0 & x < 0.\end{matrix}$$

The values of X_{U} and X_{L} are defined implicitly by the requirement that the upper pool and lower pool each contains a fraction ρ of the total population.

Three types of designs are considered: unrelated pooling designs, in which none of the 2n pooled individuals are related (although the individuals may be drawn from a larger population of related individuals); sib-together pooling designs, in which each pool consists of n/2 sib pairs; and sib-apart poolingdesigns, in which n sib pairs are split between the upper and lower pools.

Unrelated Pooling Designs

Two types of unrelated pools are shown. The first, unrelated-random, pools the n individuals with the highest and lowest phenotypic values from a population of N unrelated individuals. The term random arises because the N unrelated individuals may be obtained by selecting one sib at random from an initial population of N sib pairs.

The second unrelated design, unrelated-extreme, first reduces a population of N/2 sib pairs to N/2 unrelated individuals by selecting the individual with the more extreme phenotypic value from each sib pair. Tails with n individuals are then selected for pooling from this unrelated sub-population. The more extreme sib is defined as having a greater distance |Xⱼ| from the phenotype mean. Other definitions of distance, such as the distance from the phenotype median, or non-parametric definitions, such as the phenotype percentile score, are also possible and yield similar results for a normal distribution of phenotype scores.

Sib-Together Pooling Designs

Two sib-together designs are analyzed, each starting with a population of N individuals in N/2 sib pairs. The first, termed concordant, is analogous to concordant pooling based on a qualitative, affected/unaffected classification. If both sibs have phenotypic values above an upper threshold X_{U}, the pair is selected for the upper pool; if both values are below a lower threshold X_{L}, the pair is selected for the lower pool. The thresholds are adjusted until n/2 pairs have been added to each pool. The second sib-together design, pair-mean, is based on the phenotype mean X₊ for each pair: above X_{U} and the pair is selected for the upper pool; below X_{L} and the pair is selected for the lower pool.

Sib-Apart Pooling Designs

Two sib-apart designs are also analyzed, each starting with N/2 sib pairs. The first is termed discordant, again analogous to qualitative discordant pooling. If one sib in a pair has a phenotypic value above an upper threshold X_{U} and the other has a value below a lower threshold X_{L}, the sib with the higher value is selected for the upper pool and the sib with the lower value is selected for the lower pool. The thresholds X_{U} and X_{L} must have an additional constraint in order to arrive at a unique solution. The constraint used here is that the thresholds straddle the phenoype mean and are equidistant from it. Other constraints, such as at equal percentiles away from the median phenotype, are possible but give similar results for a normal distribution of phenotype scores.

The second sib-apart design, termed pair-difference, selects the n sib pairs with the greatest magnitude of difference |X₁−X₂| in phenotypic values. The sib with the higher value is selected for the upper pool and the sib with the lower value enters the lower pool. Again, more general measures of distance are possible.

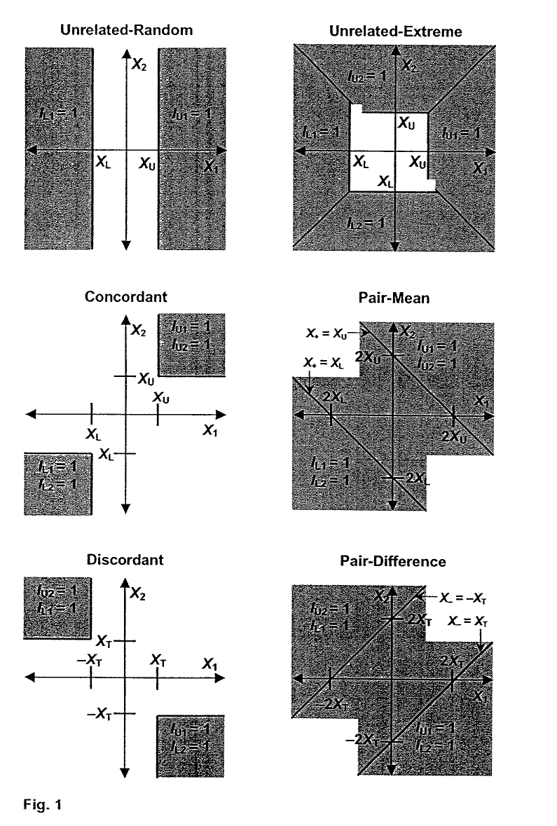

The depiction of pooling designs in FIG. 1 complements the mathematical description. Each of the six panels displays one of the pooling designs identified above. The coordinate axes are X₁ and X₂, the sib-pair phenotypic values, and cross at the overall phenotype mean of 0. Areas in the graph are shaded when one or more of the indicator functions is 1. In the unrelated-random design at the upper left, for example, an unrelated population is generated by taking the first sib from each pair and the pooled regions are vertical half-planes. If the second sib had been taken from each pair, the half-planes would be horizontal. The panel in the upper right depicts the unrelated-extreme pools. The regions corresponding to sib 1 being extreme are the two triangles bordered by X₁=±X₂ and along the horizontal axis. These regions are truncated at the upper threshold X_{U} and the lower threshold X_{L} to yield the contribution of sib 1 to the upper and lower pools. Sib 2 makes similar contributions, symmetric across the X₁=X₂ axis. This panel shows an example where X_{U}≈−X_{L}, which is the general case when the phenotype mean and median do not coincide. When equality holds, the excluded region in the center is perfectly square.

The middle panels depict the two sib-together designs. On the left is the concordant design: to be selected for pooling, both sibs must be above or below a threshold. The upper threshold X_{U} could also provide the definition for a qualitative classification affected/unaffected. In this case, the vertex of the lower pool moves northeast to meet the vertex of the upper pol at the phenotypic values X_{U},X_{U}. The panel to the right shows the pair-mean design. Here, sib pairs are selected if their mean X₊ exceeds an upper threshold X_{U} or falls below a lower threshold X_{L}. The orthogonal coordinate X₋ is uncorrelated with X₊ and unconstrained in this design. Note that the boundary lines X₊32 X_{U} and X₊=X_{L} have intercepts 2X_{U} and 2X_{L} in the X₁-X₂ coordinate system.

The bottom panels depict the discordant design on the left and the pair-difference design on the right. The discordant design selects sib-pairs from rectangular regions in the upper left and lower right; the pooling boundaries in the pair-difference design are lines of constant X₋, with X₊ unconstrained.

Despite the close analogy, there is an important difference between the concordant and discordant designs described here for quantitative traits and the designs described elsewhere for qualitative traits (Risch and Teng, 1998). In this formulation for quantitative traits, the upper and lower thresholds define tails of a population distribution and a sizeable population fraction falls between the tails. In a typical formulation for qualitative traits, and especially for qualitative traits without an obvious quantitative basis, a single threshold divides the population into two classes: a smaller affected class and a larger unaffected class holding most of the population. In the terminology used here, such designs have X_{U}=X_{L}.

2.4 Distribution of p_{U}−p_{L} Under the Alternative Hypothesis

The fraction ρ_{S} of the total population selected for each pool may be written

$${ρ}_{S} = {\sum }_{{G}_{1} {G}_{2}} {\sum }_{j = 1 , 2} {ρ}_{Sj} ( {G}_{1} , {G}_{2} ) ,$$

where, as before, S=U or L labels the upper or lower pool and

$$\begin{matrix}{ρ}_{Sj} ( {G}_{1} , {G}_{2} ) = ( 1 / 2 ) P ( {G}_{1} , {G}_{2} ) {\int }_{- ∞}^{∞} ⅆ {X}_{+} \\ {\int }_{- ∞}^{∞} ⅆ {X}_{-} f [ {X}_{+} \vert {G}_{1} {G}_{2} ] \\ f [ {X}_{-} \vert {G}_{1} , {G}_{2} ] {I}_{Sj} ( {X}_{+} , {X}_{-} ) .\end{matrix}$$

The initial factor of (½) arises because the phenotype and genotype distributions are normalized to 1 per sib-pair rather than 2. In practice, the upper and lower thresholds X_{U} and X_{L} are adjusted until the fraction in each pool is ρ<1. For an unrelated population or for a sib-pair population pooled with the pair-mean or pair-difference design, the largest possible ρ is 0.5 and the entire population splits evenly into two pools. The concordant and discordant designs have a maximum ρ that is smaller than 0.5 because, as can be seen from FIG. 1, these designs always exclude quadrants of the total population. For a sib-pair population with the unrelated-extreme design, the largest possible ρ is 0.25.

For feasible values of ρ, the expected allele frequency in pool S is

$${ρ}_{S} = {ρ}^{- 1} {\sum }_{{G}_{1} , {G}_{2} , j} {ρ}_{Sj} ( {G}_{1} , {G}_{2} ) {p}_{{G}_{j}} ,$$

where p_{G}_{ⱼ} is the allele frequency of the j^{th} sib of the pair and the expected number of such sibs selected for the pool is nρ⁻¹ρ_{S}_{ⱼ}(G₁,G₂). These numbers follow a multinomial distribution, with the following general properties: when a random variable x=n⁻¹Σᵢnᵢxᵢ with the index i ranging over a discrete set of sub-populations, the total number of samples n=Σᵢnᵢ fixed, x₁ fixed for all samples from sub-population i, the expectation values nᵢ/n=θᵢ fixed, and Σᵢθₗ=1, then the expectation value of x is Σᵢθᵢxᵢ and its variance Var(x)=n⁻¹{σᵢθᵢxᵢ²−(ρᵢθᵢxᵢ)²} (Beyer, 1984). Using these results for a multinomial distribution, the variance of the test statistic under the alternative hypothesis is written

Var(p_{U}−p_{L})=σ₁²/n

where σ₁² is independent of the number of individuals n per pool.

For the unrelated-extreme design, p_{U} and p_{L} are independent multinomial distributions and

$${σ}_{1}^{2} = {ρ}^{- 1} {\sum }_{{G}_{1} , {G}_{2} , j} \{ {ρ}_{Uj} ( {G}_{1} , {G}_{2} ) ( {p}_{{G}_{j}}^{2} - {p}_{U}^{2} ) + {ρ}_{Lj} ( {G}_{1} , {G}_{2} ) ( {p}_{{G}_{j}}^{2} - {p}_{L}^{2} ) \} .$$

For the unrelated-random design, the index j is irrelevant, yielding simpler expressions:

p_{S}=ρ⁻¹Σ_{G}ρ_{S}(G)p_{G}, and

σ₁²=ρ⁻¹Σ_{G {ρ}_{U}(G)(p_{G}²−p_{U} ²)+ρ_{L}(G)(p_{G}²−p_{U}²)}.

For the sib-together designs, I_{S1}=I_{S2} and the expected allele frequencies are

$$p s = {ρ}^{- 1} {\sum }_{{G}_{1} , {G}_{2}} 2 {ρ}_{S1} ( {G}_{1} , {G}_{2} ) {p}_{+} .$$

The corresponding frequencies θᵢ for the multinomial distribution are 2ρ⁻¹ρ_{S1}(G₁, G₂) and the effective number of samples is n/2. The resulting variance term is

$${σ}_{1}^{2} = 2 {p}^{- 1} {\sum }_{{G}_{1} , {G}_{2}} \{ 2 {ρ}_{U1} ( {G}_{1} , {G}_{2} ) ( {p}_{+}^{2} - {p}_{U}^{2} ) + 2 {ρ}_{L1} ( {G}_{1} , {G}_{2} ) ( {p}_{+}^{2} - {p}_{L}^{2} ) \} .$$

For the sib-apart designs, I_{U1}=I_{L2} and I_{L1}=I_{U2}. The expectation value of the allele frequency difference is

$$\begin{matrix}{p}_{U} - {p}_{L} = {ρ}^{- 1} {\sum }_{{G}_{1} , {G}_{2}} {ρ}_{U1} {p}_{{G}_{1}} + {ρ}_{U2} {p}_{{G}_{2}} - {ρ}_{L1} {p}_{{G}_{1}} - {ρ}_{L2} {p}_{{G}_{2}} \\ = {ρ}^{- 1} {\sum }_{{G}_{1} , {G}_{2}} 2 {ρ}_{U1} {p}_{-} + {ρ}^{- 1} {\sum }_{{G}_{1} , {G}_{2}} 2 {ρ}_{L1} ( - {p}_{-} ) .\end{matrix}$$

Due to the symmetry between the two siblings, ρ⁻¹Σ_{G}_{₁},_{G}_{₂}2ρ_{U1}=ρ⁻¹Σ_{G}_{₁},_{G}_{₂}2ρ_{L1}=1, and p_{U} −p_{L}is the sum of two multinomial distributions each with expectation value (p_{U}−p_{L})/2. The effective number of samples for each distribution is n/2, and the variance term is

$${σ}_{1}^{2} = 2 {ρ}^{- 1} {\sum }_{{G}_{1} , {G}_{2}} 2 ( {ρ}_{U1} + {ρ}_{L1} ) \{ {p}_{-}^{2} - {[ ( {p}_{U} - {p}_{L} ) / 2 ]}^{2} \} .$$

When the null hypothesis is valid, each of these expressions for σ₁ reduces to the corresponding expression for σ₀. If the alternative hypothesis is valid, σ₁ is smaller than σ₀ to the extent that variance in the test statistic is explained by the pooling design. Nevertheless, in most cases σ₀ is an excellent approximation.

2.5 Power

The statistical power 1−β to reject the null hypothesis for a single one-tailed test with p-value αwhere α is equivalent to the false-positive rate or Type I error rate and β is equivalent to the false-negative rate or Type II error rate, is

1−β1−Φ{[z_{α}σ₀−\sqrt{n}(p_{U}−p_{L})]/σ₁},

where Φ(z) is the cumulative standard normal distribution, 1−Φ(z_{α})=α. Solving for n and using the relation n/N=ρ, the total number of individuals N necessary to generate pools with the required power is

N=ρ⁻¹[(z_{α}σ₀−z_{1-β}σ₁)/(p_{U}−p_{L})]²,

where ρ=n/N is the fraction of the total population selected for each pool. In either case, replacing σ₁ with σ₀ would result in a conservative test.

2.6 Computational Methods

Exact results for the distribution of the test statistic T under the null hypothesis and under the alternative hypothesis, subject only to the approximation that T is normal, were obtained by numerical computations converged to better than 1 part in 10⁶ (Press et al. 1997). Brent's root-finding algorithm was used to determine the threshold values X_{U} and X_{L} for the upper and lower pools for a given pooling design and pooling fraction ρ, Brent's optimization algorithm was then used to find the ρ with maximum power. The integrals providing p_{U} −p_{L} and σ₁² were evaluated numerically using Romberg integration with a change of variables to the reciprocal for infinite integration limits. Integration was restricted to regions where an indicator function was non-zero. In order to reduce computational requirements, the final integral of a normal distribution over fixed limits was evaluated using a polynomial approximation to the error function. This technique reduced the two-dimensional integrals over bivariate normals to one-dimensional integrals for the unrelated-extreme, concordant, and discordant designs, while integration was avoided completely for the unrelated-random, pair-mean, and pair-difference designs. The 9 sib-pair genotypes were reduced by symmetry to 5 genotypes for further savings. Using a 750 MHz Pentium III running Linux, the root-finding and minimization for each parameter set required less than 0.01 see each for the unrelated-random, pair-mean, and pair-difference designs and approximatley 6 sec each for the unrelated-extreme, concordant, and discordant designs.

The numerical results, and the underlying theory, are robust when n, the number of individuals per pool, is large and 2(p_{U}+p_{L})n, the number of alleles in the pools, approximately follows a normal distribution. In certain regions of extreme parameter values, however, the numerical solution for n drops below 1. This behavior signals a breakdown of various assumptions of the theory, and results in these regions are unreliable.

The properties and characteristics of the methods of the present invention are set forth in the Examples. It is shown, for example, that the optimal design for unrelated individuals is to pool the top and bottom 27% of the population. This design using N unrelated individuals has greater power than designs using N/2 sib pairs when the phenotypic correlation between sibs is low to moderate, below 75%, but has less power than sib pair designs when the correlation is above 75%.

Of the designs explored for a population of sib pairs, the unrelated-extreme design is the best for low to moderate sibling phenotype correlation. In this design, the more extreme sib is selected from each pair, then the top and bottom 36% of this subset are pooled. When the correlation is high, above 75%, the best design found for sib pairs is to first select the 27% of pairs with the greatest phenotype difference, then split each pair by phenotypic value to form an upper and lower pool. The pair-difference design might also be applied at low to moderate sibling correlation to reduce the rate of spurious association due to population stratification. The optimal pooling fractions for these designs were determined by minimizing the population requirements. The minima were generally quite flat, and pooling fractions close to the optimal fractions give near-optimal results.

Compared with the results obtained by others for pooling based on qualitative traits, the results derived using the methods of the present invention for quantitative traits are thought to be surprising. For earlier pooling strategies based on qualitative traits, designs using unrelated individuals were found to be more powerful than designs using sib pairs; when populations were restricted to sib pairs, concordant designs were found to have greater power than discordant designs (Risch and Teng 1998). In contrast, for quantitative phenotypes, the methods of the present invention indicate that unrelated individuals become less powerful than sib pairs when sibling correlation is high, and that sib-apart designs become more powerful than sib-together designs when the sibling correlation is above 50%. This result is significant because highly heritable traits that are likely to be the first targets of large-scale genotyping studies often exhibit sibling correlations of 50% or higher. Quantitative phenotypic values also permit the use of the unrelated-extreme design, which does not have an obvious analog for qualitative phenotypes that categorize individuals as affected/unaffected.

The sib-together and sib-apart pooling designs of the present invention, which draw individuals from extreme-high and extreme-low phenotypes, are anticipated to be more powerful than alternative designs that compare one extreme to the remainder of the population, as in a qualitative affected/unaffected classification. The affected/unaffected classification establishes a single threshold for a quantitative phenotype, and the allele frequency in the large unaffected class is close to the population mean. In contrast, the quantitative designs of the present invention employ two thresholds, and the allele frequencies in the upper and lower pools are approximately equidistant from the population mean. The allele frequency difference between pools is consequently half as large for the qualitative design as for the quantitative design of the present invention, and the population requirements are four times as large, or half as large if the overall allele frequency is assumed to be known exactly. These conclusions are similar to those reached in the context of linkage analysis for quantitative trait localization using extremely concordant and extremely discordant sib pairs (Risch and Zhang 1995, Risch and Zhang 1996, Zhang and Risch 1996, Gu et al. 1996).

As with most genotyping designs, the pooling strategies described here are primarily sensitive to the additive variance from an allele. Since the additive variance for an allele is approximately equal to the fraction of heterozygotes times the square of half the phenotype shift between the two homozygotes, rare alleles with larger phenotype shifts may be detected with the same power as common alleles with smaller shifts. When the allele frequency becomes smaller than the additive variance of the allele, however, the frequency shift must become very large to compensate and the phenotype begins to resemble a monogenic trait.

The results provided here also imply the precision required for allele frequency determinations for pooled DNA. Approximately 3000 individuals are required for a genome-wide screen with an optimal pool size n of 600 to 800 individuals. The frequency difference corresponding to significance at α=5×10⁻⁸ (z_{α}=5.33) for a polymorphism with minor-allele frequency p₁ is z_{α}[p₁(1−p₁)/n]^{1/2}, which is 5% for an allele frequency of 0.1 and 2% for an allele frequency of 0.01. An experimental measurement should provide an order of magnitude better precision in the allele frequency difference to avoid losing information.

3. Examples for Model 1

Overview to the Examples

In this section, total population sizes are presented for a wide range of parameters and as functions of the pooling fraction ρ. The first parameters explored are the sib-pair phenotype correlation r and the allele frequency p₁; these parameters are readily determined experimentally at the start of an association study. The next set of parameters explored are the additive phenotype variance σ_{A}², the dominance ratio d/a, and the resulting dominances variance σ_{D}² and genotype effects μ_{G}, which are not known at the start of a study. Finally, the dependence of the population requirements on the false-positive rate α and false-negative rate β is explored. As each single parameter is varied in turn, the remaining parameters are held fixed at a set of values selected to serve as a common reference.

The reference value for sibling phenotype correlation was based on reported values for genetic heritabilities and shared environmental factors. Estimates of the genetic heritability for complex traits range from 20% for cancer (Verkasalo et al. 1999), 20% to 40% for Type 2 diabetes mellitus (NIDDM) (Watanabe et al. 1999), 50% for pulmonary function (Wilk et al. 2000), 10% to 50% for systolic and diastolic blood pressure (Iselius et al. 1983, Perusse 1989), and 70% to 90% for cholesterol level (Austin et al. 1987). Shared environmental factors are estimated to contribute 7% of the overall phenotype variance for cancer (Verkasalo et al. 1999), 20% to 40% for blood pressure (Iselius et al. 1983, Perusse et al. 1989), and 15% for serum lipid levels (Heller et al. 1993). The sibling phenotype correlation, equal to half the genetic heritability plus the shared environmental contribution, varies over a wide range for these traits. A phenotype correlation of 40%, in the middle of the range, was selected to serve as the reference.

Reported minor-allele frequencies for SNPs found in multiple populations range from 5% to 25%, with lower frequencies for variations which cause non-conservative amino acid changes and higher frequencies for conservative substitutions and changes in non-coding regions (Cargill et al. 1999, Goddard et al. 2000). A reference value of 10% was selected for p₁, typical of changes in the coding region.

The genetic variance arising from a typical SNP was modeled by assuming that the genetic heritability arises from multiple loci, each of which makes an independent contribution with a characteristic size equal to the genetic heritability divided by the total number of contributing loci. Assuming that approximately 20 polymorphic sites contribute to a genetic heritability of 40% yields a reference value of 0.02 for σ_{A}²+σ_{D}². The reference value selected for the dominance ratio was d/a=0, indicating a purely additive allele.

In practice, the false-positive rate α is matched to the number of individual tests that are to be conducted in an association study. For a genome scan of 10⁶ individual markers versus a single phenotype, for example, or for a scan of 10⁴ markers versus 100 distinct phenotypes, a false-positive rate α per marker should be no more than 5×10⁻⁸ for a final p-value <0.05 for the detection of an association. If only 1000 markers are used, for example as in a test of candidate polymorphisms, then the value α=5×10⁻⁵ suffices. The false-positive rate selected as a reference was α=5×10⁻⁶ (z_{α}=5.33), a value suggested to provide a sufficiently low number of false positives after applying a multiple-hypothesis-testing correction corresponding to a full-genome scan (Risch and Merikangas 1996). The power 1−β was fixed at 0.8 (z_{1-β}=−0.84) for a 20% false-negative rate.

Figures depicting the results use a consistent scheme. The unrelated designs are represented as solid lines, thin for unrelated-random and thick for unrelated-extreme; the sib-together designs are represented as equal-spaced dashed lines, thin for concordant and thick for pair-mean; and the sib-apart designs are represented as unequally-spaced dashed lines, thin for discordant and thick for pair-difference.

### EXAMPLE 1

### Sibling Phenotype Correlation

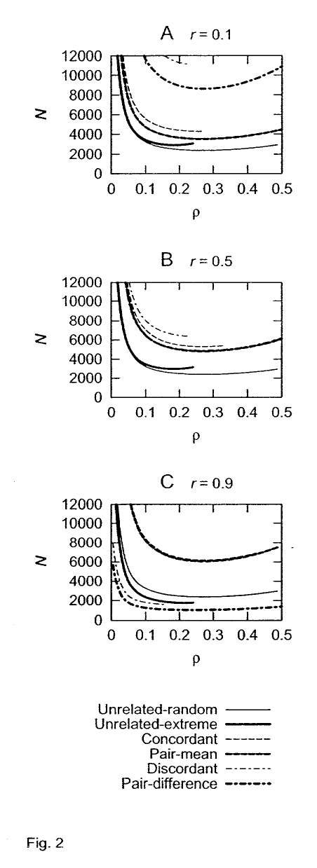

The minimum population size N required to detect association as a function of the sibling phenotype correlation r and the pooled fraction ρ is shown in FIG. 2, with the remaining parameters at their previously defined reference values (α=5×10⁻⁸, 1−β=0.8,p₁=0.1, σ_{A}²=0.02, d/a=0). The three panels in FIG. 2 show a range of sibling phenotype correlations: r=0.1 (Panel A), 0.5 (Panel B), and 0.9 (Panel C). In each panel, as the pooling fraction increases from ρ=0, each design has a sharp then more gradual decrease in population requirements. Eventually N attains a minimum, indicating the optimal pooling fraction for maximum power, and then gradually increases with ρ. A second feature seen in all three panels is the similarity between the unrelated designs, between the sib-together designs, with pair-mean always more powerful than concordant, and between the sib-apart designs, with pair-difference always more powerful than discordant. Furthermore, for larger values of ρ the required numbers of concordant and discordant sib pairs are not met.

In FIG. 2, Panel A shows that for small values of the phenotype correlation the design with the greatest power is unrelated-random, with unrelated-extreme slightly less powerful. The sib-together designs require approximately twice as large a sample, and the sib-apart designs require three to four times as many. In Panel B, at the intermediate phenotype correlation r=0.5, the unrelated designs are still the most powerful, while the sib-together designs have increased population requirements and the sib-apart designs have decreased to meet in the middle. At large values of the sibling phenotype correlation, r=0.9 in Panel C, the sib-apart designs are most powerful. The unrelated designs require approximately twice as large a population, and the sib-together designs have far greater requirements.

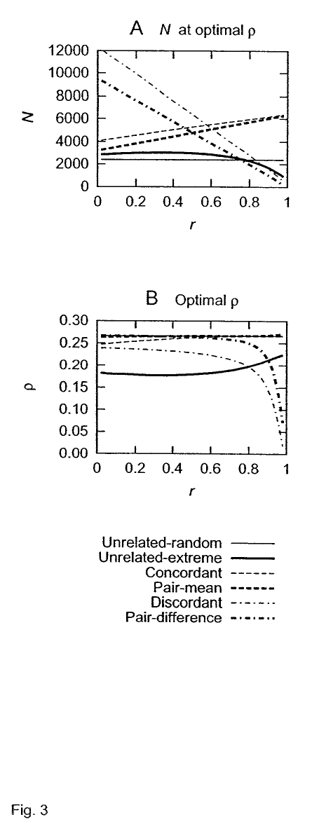

The regions near the minima of N for each design are quite flat, indicating that pooling fractions within 0.1 of the minimum may give near-optimal results. The exact values; of these minima are depicted in FIG. 3. The population requirements are shown in Panel A, and the corresponding optimal pooling fractions are shown in Panel B. The unrelated-random design is insensitive to the sibling correlation r, as seen in Panel A, as is the unrelated-extreme design except at the highest values of r. The sib-together designs require larger populations as r increases, while the sib-apart designs require smaller populations. The sib-together and sib-apart designs cross near r=0.5, and the sib-apart and unrelated designs cross near r==0.75. The optimal pooling fractions are insensitive to the changes in the sibling correlation for values below r=0.75, as seen in Panel B. The unrelated-random, pair-mean, pair-difference, and concordant designs have an optimal ρ near 0.27 in this region of low to moderate correlation, while the unrelated-extreme design has an optimum near ρ=0.18 and the discordant design near 0.23. For phenotypes with high correlation, r>0.75, the optimal fraction for ρ decreases and only highly discordant sibs are selected for the sib-apart designs.

### EXAMPLE 2

### Allele Frequency

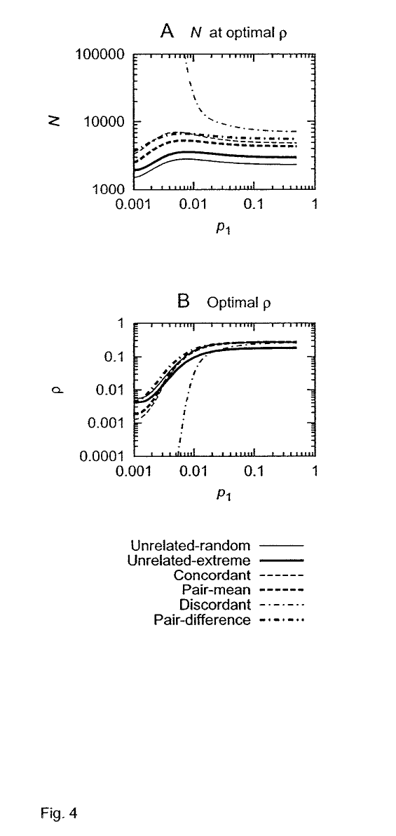

The results of changing the allele frequency p₁ while optimizing the pooling fraction and holding the remaining parameters constant at their reference values (α=5×10⁻⁸, 1−β=0.8, r=0.4, σ_{A}²=0.02, d/a=0) are shown in FIG. 4. The population requirements corresponding to the optimal pooling fraction ρ are shown in Panel A, and the corresponding fractions ρ in Panel B. The dependence ρ₁ is symmetric about p₁=0.5; results are shown only for the region p₁<0.5 and are displayed on a logarithmic scale to highlight the behavior at low allele frequency.

At moderate frequencies of the minor allele, p₁>1%, the power and pooling fraction are both insensitive to the allele frequency. This behavior, which arises when σ_{A}² is held constant and changes in μ_{G} are allowed to compensate for changes in p₁, is often observed in variance components models (Liu 1997). Thus, as long as the allele frequency is not too small, lower frequency alleles with larger effects and higher frequency alleles with smaller effects are found with similar power.

At smaller allele frequencies, p1<1%, the increasingly rare allele has an corresponding large effect μ_{G} on the phenotype, and the population requirements decrease. The crossover into this region occurs when the allele frequency p₁ falls below its contribution σ_{A}²+σ_{D}² to the overall phenotypic variance. The pooling fraction also decreases with p₁ in this region. The exception to this trend is the discordant design, which has a dramatic drop in power for low frequency alleles.

### EXAMPLE 3

### Additive Allele Variance

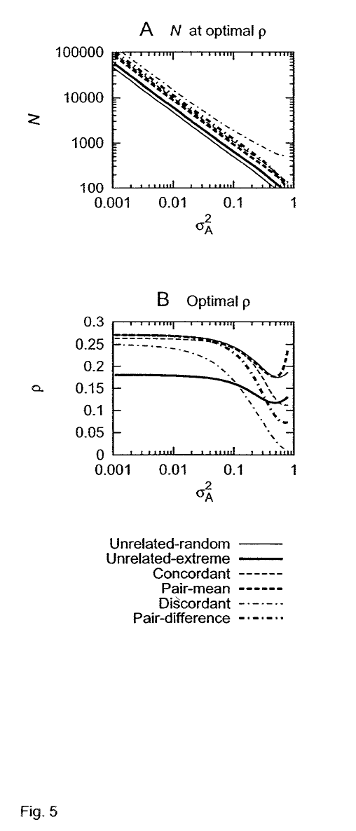

The population size N required to detect association is shown as Panel A in FIG. 5 as a function of the additive phenotypic variance arising from genotype G, with the remaining parameters fixed at their reference values (α=5×10⁸, 1−β=0.8, r=0.4, p₁=0.1, d/a=0). The population size and the variance have a clear inverse linear relationship over three orders of magnitude. This behavior corresponds to N∝(p_{U} −p_{L})⁻² with p_{U} and p_{L} proportional in turn to σ_{A}.

The corresponding optimal pooling fractions are shown in FIG. 5, Panel B. Over most of the range, σ_{A}²<0.1, the optimal fractions are not sensitive to the variance arising from the allele. At larger values of the variance the phenotype becomes nearly monogenic and smaller pooling fractions and populations are required.

### EXAMPLE 4

### Recessive, Additive, and Dominant Alleles

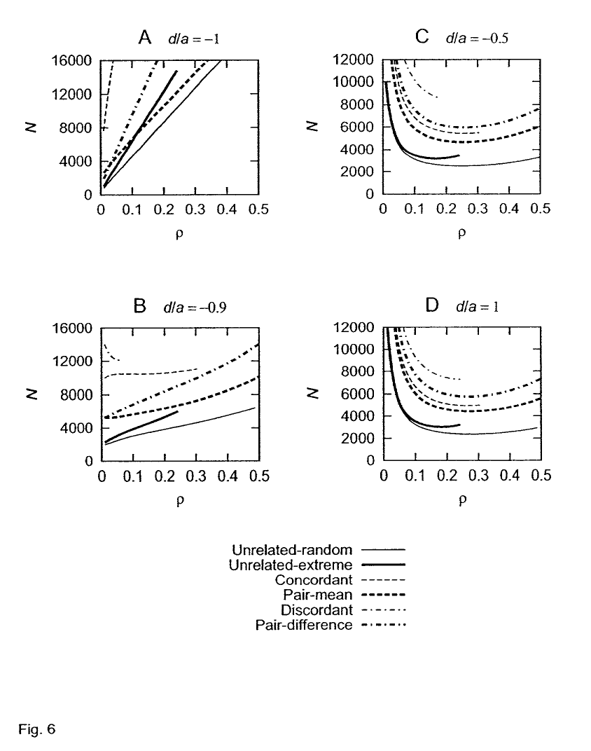

The series of panels in FIG. 6 depicts the required population size as a function of the pooling fraction ρ for a range of dominance ratios d/a. The values for d/a were selected to provide adequate sampling of the ratio of the dominance variance to the additive variance. This contribution, σ_{D}²/(σ_{A}²+σ_{D}²), is 82% at d/a=−1 (pure recessive), 65% at −0.9, 11% at −0.5, and 5% at +1 (pure dominant). The remaining parameters were held at their reference values (α=5×10⁻⁸, 1−β=0.8, r=0.4, p₁=0.1, σ_{A}²=0.02, d/a=0). The pooling fraction was set to ρ=0.2 for this series of panels and represents a near-optimal fraction for additive variance, d/a=0.

For pure recessive traits, d/a=−1 in Panel A (82% dominance variance for p₁=0.1), the estimate for N approaches an apparent minimum at ρ=0, and the assumption of normality of the test statistic is no longer valid. When d/a is −0.9 and the dominance variance contribution has dropped to 65%, the curves for N in Panel B start to flatten, and when d/a=−0.5, in which the heterozygote mean is still three-quarters of the way towards the minor-allele homozygote, the curves in Panel C are nearly indistinguishable from the results for pure additive (not depicted) and pure dominant, Panel D.

These results again signal that pooling methods for quantitative phenotypes are more sensitive to changing additive variance than to changing dominance variance. The dominance variance is only significant in regions where the additive variance vanishes, d/a=1/(p₁−p₂). This region occurs near −1 for a low-frequency allele, indicating that association studies have weak power to detect low-frequency recessive alleles or their high-frequency dominant counterparts.

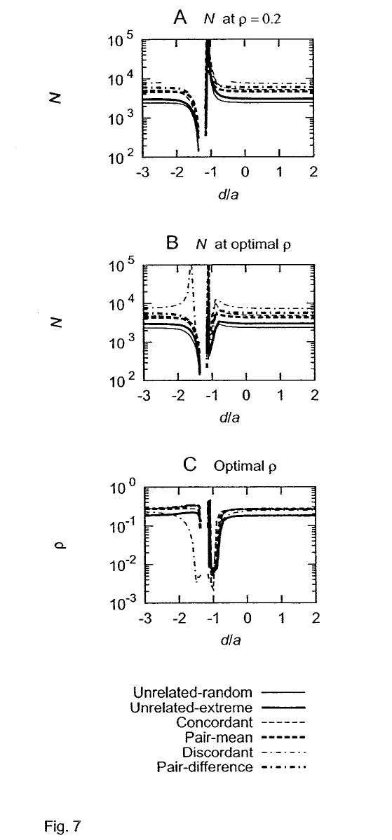

These effects are shown in greater detail in FIG. 7. The population requirements are shown as a function of the dominance ratio d/a at the fixed pooling fraction ρ=0.2 in Panel A and at the optimal fraction in Panel B. Other than the region in which the additive variance vanishes, d/a=−1.125 for p₁=0.1, the results in both panels are similar and show little dependence on d/a. This is true even in regions of strong over-dominance, d/a>1, and under-dominance, d/a<−2. Near the region of vanishing additive variance the optimal pooling fraction ρ drops rapidly, as seen in Panel C, and the results for the optimal ρ and ρ=0.2 differ.

### EXAMPLE 5

### False-Positive Rate and False-Negative Rate

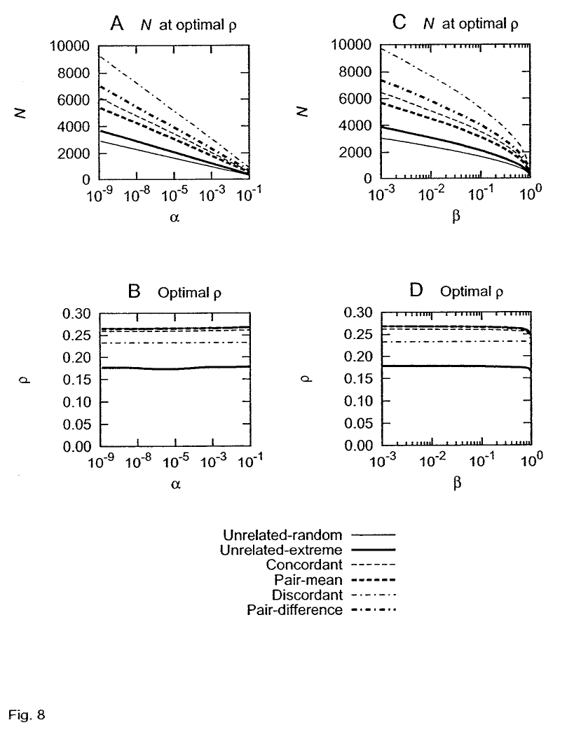

When the widths of the distribution of the test statistic under the null and alternative hypothesis are approximately equal, the equation for the population necessary to detect association has the form N∝(z_{α}−z_{1-β})². When a becomes small, the behavior z_{α}˜[−2 ln(α)]^{1/2} for small α, extracted from an asymptotic expansion for Φ(z) (Mathews and Walker 1970), leads to the asymptotic behavior N˜2 ln(1/α), which is seen clearly as the linear behavior in Panel A of FIG. 8. The remaining parameters are fixed at their reference values (1−β=0.8, r=0.4, p₁=0.1, σ_{A}²=0.02, d/a=0). Compared to a whole-genome scan with α=5×10⁻⁸ (z_{α}=5.33) and a 20% false-negative rate, for example, which requires 2400 individuals pooled with the unrelated-random design or 3000 siblings pooled with the unrelated-extreme design, a test of 1000 candidate polymorphisms with α=5×10⁻⁵ (z_{α}=3.89) requires 1400 unrelated individuals or 1800 siblings, while a test for association between a single polymorphism and a single phenotype, α=0.05 (z_{α}=1.64), require 400 unrelated individuals or 500 siblings. The optimal fraction ρ for pooling is not sensitive to the choice for α itself, as seen in Panel B.

The effects of varying the false-negative rate β are similar to the effects of varying α because the population requirements depend predominantly on the difference z_{α}−z_{1-β} rather than on the value of either alone. For small values of β, N˜2 ln(1/β). This linear behavior is demonstrated in Panel C, where the remaining parameters have their reference values except for α=5×10⁻⁵ corresponding to a test of candidate polymorphisms. The optimal pooling fraction ρ does not depend sensitively on β, as shown in Panel D.

4. Model 2

4.1 Variance Components Model

A standard variance components model is used to describe the joint phenotype-genotype probability distribution. A quantitative phenotype X, standardized to mean 0 and variance 1, is hypothesized to be affected by the genotype G at a biallelic locus with minor allele A₁ and major allele A₂ occurring at population frequencies p and 1−p. More generally, A₂ may represent any of a number of alternate alleles, and 1−p their aggregate frequency. The population is assumed to be random mating and in Hardy-Weinberg equilibrium. The symbol P is used to denote a probability, and the genotype frequencies P(G) are p², 2p(1−p), and (1-p)² for A₁A₁, A₁A₂, and A₂A₂ respectively. The frequency of allele A₁ in genotype G, denoted p_{G}, is 1 for A₁A₁, 0.5 for A₁A₂, and 0 for A₂A₂. The variance of the allele frequency for an individual, denoted σₚ², is p(1−p)/2.

The frequency of a genotype combination for a sib pair is denoted P(G₁,G₂). Only full sibs are considered. The probability distribution P(G₁,G₂) of the 9 possible combinations of sib-pair genotypes, shown in Table III, can be derived by considering all possible parental mating types and their offspring genotype distributions [ ] (i. Neale, M C and Cardon, L R: Methodology for Genetic Studies of Twins and Families; in NATO ASI Series D, Behavioural and Social Sciences, vol 67. Dordrecht, Kluwer Academic, 1992).

The effects μ(G) of genotype G are to displace the phenotypic mean by a, d, and −a for genotypes A₁A₁, A₁A₂, and A₂A₂ respectively, with the raw mean (2p−1)a+2p(1−p)d then subtracted to preserve the overall phenotypic mean of 0. The relationship between d and a determines the inheritance mode of allele A₁: d=−a for a recessive allele, +a for a dominant allele, and d=0 for an additive allele.

The phenotypic variance contributed by the genotype G can be partitioned into an additive component σ_{A}² and a dominance component σ_{D}², with

σ_{A}²+σ_{D}²=2p(1−p)[a−d(2p−1)]²+4p²(1−p)²d².

As will be seen below, this partitioning is important because association tests are sensitive primarily to σ_{A}², not to σ_{D}². Note that σ_{A}² may be much larger than σ_{D}² even when the inheritance is purely dominant or recessive. Remaining genetic and environmental factors contribute a residual variance σ_{R}²=1−(σ_{A}²+σ_{D}²) to the total phenotypic variance.

The probability density of phenotypic values for sib pairs is denoted f(X₁,X₂). It can be expressed as a mixture of 9 conditional densities, one for each possible sib-pair genotype,

$$f ( {X}_{1} , {X}_{2} ) = {\sum }_{{G}_{1} {G}_{2}} f ( {X}_{1} , {X}_{2} \vert {G}_{1} , {G}_{2} ) P ( {G}_{1} , {G}_{2} ) .$$

The mean of X₁ is μ(Gᵢ) for sib i=1 or 2; both X₁ and X₂ have residual variance σ_{R}² and residual covariance (due to shared residual genetic and environmental factors) t_{R}. The total phenotypic correlation t for sib pairs is

t=t_{R}+σ_{A}²/2+σ_{D}²/4

when effects from genotype G are included.

Although X₁ and X₂ are natural coordinates for expressing sib phenotypic values, the correlation between sibs complicates the joint distribution of X₁ and X₂. A simpler joint distribution is obtained by noting that the sum and difference of X₁ and X₂ are completely uncorrelated. These orthogonal coordinates representing sib mean and sib difference are denoted X₊ and X₋, with

X_{±}=(X₁±X₂)/2.

The probability distribution in these orthogonal coordinates, f(X₊,X₋|G₁G₂), factors into the product off j(X₊|G₁,G₂) and j(X₋|G₁,G₂), with

f(X_{±}|G₁,G₂)=(2πσ_{±}²)^{-1/2}exp{−[X_{±}−μ_{±})(G₁,G₂)]²/2σ_{±}²}, using

μ_{±}(G₁,G₂)=[μ(G₁)±μ(G₂)]/2 and

σ_{±}²=σ_{R}²(1±t_{R})/2.

It is also convenient to define pair-mean and pair-difference allele frequencies p±(G₁,G₂) as p±(G₁,G₂)=(p_{G}_{₁}±p_{G}_{₂})/2.

The variance of the pair-mean and pair-difference variables may be expressed more generally for sib-ships of size s, with genotypic correlation r between any two sibs within a sib-ship, as

Var(X_{±})=σ_{R}²T_{±} and

Var(p_{±})=σₚ²R_{±}

where

T_{±}=[1±(s−1)t_{R}]/S and

R_{±}=[1±(s−1)r]/s.

The family size s is 2 for sib-pairs, and the genotypic correlation r is 0.5 for full sibs.

In addition to X₁,X₂ and X₊,X₋ coordinate systems, we also introduce a Mahalanobis coordinate system. In this metric, a sib-pair is described by a radial coordinate b, which expressed how extreme the pair of phenotypic values is, and an angle φ, which determines whether each sib has a positive or negative phenotypic value. The transformations relating the Mahalanobis variables to the pair-mean and pair-difference variables are

X₊=σ₊b sin φ and

X₋=σ₊b cosφ.

The probability distribution in Mahalanobis coordinates is

f(b,φ|G₁,G₂)=(2π)⁻¹exp[−(b sinφ−ν+)₂/2]exp[−(b cosφ−ν₋)²/2] with

ν_{±}=μ_{±}/σ_{±}.

This distribution satisfies

$${\int }_{0}^{2 π} ⅆ ϕ {\int }_{0}^{∞} ⅆ b b f ( b , ϕ \vert {G}_{1} , {G}_{2} ) = 1.$$

In the absence of a contribution from the QTL,f(b,φ|G₁,G₂) reduces to (2π)⁻¹ exp(−b²/2) and the Mahalanobis probability density is independent of the phase φ. Contour lines of equal probability density in the X₁−X₂ plane are ellipses tilted at 45° with a ratio of major axis to minor axis of [(1+t)/(1−t)]^{1/2}.

4.2 Test Statistic and Pool Design

The tests of association described here depend on detecting differences in allele frequency in DNA pooled from individuals chosen from a large repository DNA repository. The allele frequency in the upper pool, with individuals selected to have higher phenotypic values, is denoted p_{U}; the allele frequency in the lower pool, selected for lower phenotypic values, is p_{L}; and the test statistic is p_{U}−p_{L}, denoted Δp.

The overall repository size is denoted N, composed entirely of either N unrelated individuals or N/2 sib pairs. The upper and lower pools each hold n samples, and the pooling fraction ρ is defined as n/N.

For an unrelated population, only one design is described: selecting the n individuals whose phenotypic values are at the upper and lower tails of the distribution, thus defining upper and lower thresholds X_{U} and X_{L}. This is termed the unrelated-population design.

A corresponding design for sib pairs is termed unrelated-random. In this design, one sib is chosen, at random, from each sib-ship to generate a population of N/2 unrelated individuals. Individuals at the upper and lower tails of this unrelated subset are then selected for pooling. The unrelated-random design for N/2 sib pairs with pooling fraction ρ is essentially equivalent to the unrelated-population design for N/2 individuals with pooling fraction 2p.

A second design selecting only unrelated individuals is termed the Mahalanobis design. The pair-mean X₊ and pair-difference X₋ are used to define a Mahalanobis coordinate b according to

b₂=2X₊²/(1+t)+2X₊²/(1−t).

The n sib-ships with the largest magnitude b and a positive pair-mean X₊ are identified, and the sibling with the larger phenotypic value is selected for the upper pool. Similarly, the n sib-ships with the largest b and negative pair-mean are identified, and the sibling with the more negative phenotypic value is selected for the lower pool.

Two remaining designs select both members of a sib pair for pooling. The pair-mean design selects each sib-ship as a family unit based on the phenotypic mean of the pair. The n/2 pairs at the extreme upper and lower tails of the distribution of phenotypic means for sib-ships, comprising n individuals each, are selected for the upper and lower pools respectively The upper and lower thresholds are again termed X_{U} and X_{L}.

The pair-difference design selects individuals based on the difference of phenotypic values within each sib-ship, or equivalently on the magnitude of within-family phenotypic variance. The n sib-pairs with the greatest within-family variance are identified. Within each pair, the individual with the higher phenotypic value is selected for the upper pool, and the individual with the lower phenotypic value is selected for the lower pool. The threshold for the magnitude of the difference |X₁−X₂|/2 for selecting families is termed X_{T}.

Since the X₊ and X₋ are uncorrelated within each family, the results of the pair-mean and pair-difference designs are statistically independent and may be combined to yield a single, potentially more powerful test.

4.3 Test Power

Under the null hypothesis H₀, the expectation for p_{U} and p_{L} is the population mean allele frequency, and the expectation for the test statistic Δp is zero. Under the alternative hypothesis H₁, the expectation E₁(Δp) for Δp is non-zero. The power of a test of Δp depends on the magnitude of E₁(Δp) compared to the variation of Δp under H₀ and H₁, and in turn on the variation of p_{U} and p_{L}.

Both p_{U} and p_{L} follow multinomial distributions defined by the probability that an individual with zero, one, or two copies of allele A₁ is selected for pooling. When the total number of individuals selected for each pool is large and the number of copies of allele A₁ in each pool is also large, the multinomial distribution giving Δp is described accurately by a normal distribution. The variance of Δp under H₀ is denoted σ₀²/n and the variance under H₁ is denoted σ₁²/n, where σ₀² and σ₁² depend on the model parameters and the pooling design. The number of individuals required for type I error rate α and type II error rate β is

n=(z_{α}σ₀−z_{1-β}σ₁)²/E₁(Δp)².

The terms z_{α} and z_{1-β} are normal deviates corresponding to the error rates,

Φ(z_{α})=1−α, and Φ(z_{1-β})=β,

where Φ(z) is the cumulative probability function for the standard normal distribution,

$$Φ ( z ) = {\int }_{- ∞}^{z} ⅆ {z ( 2 π )}^{- 1 / 2} exp ( - {z}^{2} / 2 ) .$$

The significance level α is for a one-sided test, which is appropriate for association tests for disease-susceptibility markers. If markers for protective polymorphisms are also sought, the significance for a two-sided test is more appropriate.

The method used here to optimize test designs is to specify the error rates α and β, then calculate the selection criteria that minimize the total repository size N required to achieve these error rates for specific genetic models. The method is outlined below, along with a summary of analytical approximations for the repository sizes required for different population structures and pooling designs. Comparisons of the analytical approximations with essentially exact numerical calculations are found in the Results section, and mathematical details are provided in the Appendix.

To optimize N, a trial value of the fraction ρ is chosen. Next, the threshold phenotypic values that select n=ρN individuals for each pool are derived from the distribution of phenotypic values. Depending on the pooling design, these threshold values may refer to phenotypes for unrelated individuals, the Mahalanobis measure b, the pair-mean measure X₊, or the pair-difference measure X₋. The threshold values are used to calculate the probabilities θ_{U}(G) and θ_{L}(G) that an individual selected for the upper and lower pools has a particular genotype G. These probabilities provide the expectation E₁(Δp) of Δp under H₁, as well as the variances σ₀²/n and σ₁²/n of Δp under H₀ and H₁. Values of Δp larger than z_{α}σ₀/n^{1/2} are significant at level α, and the corresponding power of the test is

1−β=Φ{[ρ^{1/2}N^{1/2}E₁(Δp)−z_{α}σ₀]/σ₁}.

Since the terms E₁(Δp), σ₀², and σ⁰² depend on ρ but not on N or n, this equation may be inverted to find N as a function of 1−β,

N=(z_{α}σ₀z_{1-β}σ₁)²/ρE₁(Δp)².

Optimization proceeds by a search for the value of ρ giving smallest N.

For complex traits, the total variance σ_{A}²+σ_{D}² from any QTL is small, and σ_{R}² is close to 1. This suggests that the displacements a, d, and −a are also small relative to σ_{R} and motivates a perturbation expansion of E₁(Δp) and σ₁² in terms of μ(G)|σ_{R}. The expression for Δp is linear in the expansion parameter, while σ₁² is identical to σ₀² to first order. Collecting the lowest order terms, the result for the required repository size N is proportional to σ_{R}²/σ_{A}². The constant of proportionality depends on the pooling fraction ρ, the phenotypic correlation between sibs, the specified values of α and β, and the pooling design, but not on any properties of the genetic model other than σ_{A}².

In deriving the optimal test designs and estimating the test power, we assume implicitly that there is no measurement error in either the allele frequency p or the allele frequency difference Δp. For the allele frequency p, we show in the Results that either using the mean value (p_{U}+p _{L})/2 or measuring the allele frequency on a large pool of individuals unselected for phenotypic value should provide an adequate estimate for p. We also discuss the reduction in power due to measurement error in Δp.

Unrelated Design

When a repository contains N unrelated individuals, the analytical approximation for the required repository size, derived in the Appendix, is

N_{urelated}=(ρ/2yₚ²)(z_{α}−z_{1-β})²σ_{R}².

This functions is a minimum at ρ=0.27, with ρ/2yₚ²=1.24.

If the population consists of sib pairs rather than unrelated individuals, an unrelated sub-population of N/2 individuals may be constructed by selecting one sib at random from each pair. A direct extension of the above result for unrelated populations yields

N_{random-sib}=2[(2ρ)/2y₂_{ₚ}²](z_{α}−z_{1-β})²σ_{R}²/σ_{A}²

for the sib-pair population. The repository size required for sib pairs is twice as large as for unrelated individuals, with a pooling fraction half as large.

Mahalanobis Design

The analytical approximation for the number of individuals required for the Mahalanobis design, derived in the Appendix, is

N_{Mabal}=(2ρ)⁻¹[(2b_{ρ}/π)+Φ(−b_{ρ)/ρ(}2π)^{1/2}]⁻²[R₊/T₊^{1/2}+R₋/T₊^{1/2}]⁻²(z_{α}−z_{1-β})²σ_{R}²/σ_{A}².

The initial geometrical factor depends only on the pooling fraction. It is minimized at ρ=0.188 with a value of 2.90, yielding

N_{Mahal}=2.90[R₊/T₊^{1/2}+R₋/T₋^{1/2}]⁻²(z_{α}−z_{1-β})²σ_{R}²/σ_{A}²

for this pooling design.

Pair-Mean Design

The analytical approximation for the repository size required by the pair-mean design is

Nₚₐᵢᵣ₋ₘₑₐₙ=(sρ/2y_{ρ}²)(T₊/R₊)(z_{α}−z_{1-β})²σ_{R}²/σ_{A}²,

where s=2 for sib pairs. As with the unrelated design, the factor ρ/y_{ρp}² is optimized with a pooling fraction of 0.27, yielding

Nₚₐᵢᵣₘₑₐₙ=2.47(T₊/R₊)(z_{α}−z_{1-β})²σ_{R}²/σ_{A}²

for the required repository size.

Pair-Difference Design

An analytical approximation for the repository size required by the pair-difference design is

N_{pair-diff}=(sρ/2y_{ρ}²)(T₋/R_)(z_{α}−z_{1-β})²σ_{R}²/σ_{A}²

The factor ρ/y_{ρ}² is minimized with a pooling fraction of 0.27, and

N_{pair-diff}=2.47(T₋/R₋)(z_{α}−z_{1-β})²σ_{R}²/σ_{A}²

is the required repository size.

Combined pair-mean and pair-difference design Because the sib-mean variables X₊ and p₊ are uncorrelated with the pair-difference variables X₋ and p₋, the pair-mean and pair-difference estimators are independent and may be combined into a single test. The combined test uses the measured value of Δp_{±}, where the + and − signs refer to the allele frequency differences found for the pair-mean and pair-difference pools, to obtain an estimator for σ_{A}/σ_{R}, The pair-mean and pair-difference estimators, Q_{±}, each with expectation σ_{A}/σ_{R}, are

Q_{±}=(T_{±}^{1/2}/R_{±})(σ/2y_{ρ}σ_{ρ})Δp_{±}, with

Var(Q_{±})=(sρ/2y_{ρ}²N)T_{±}/R_{±}

from the expressions provided in the Appendix for Var(Δp_{±}). These expressions differ by the factor sR_{±} from a similar expression provided by Ollivier et al. (1997) which incorrectly neglected the contribution of family structure to Var(Δp_{±}).

The combined minimum-variance estimator Q having expectation σ_{A}/σ_{R}, constructed by weighting the pair-mean and pair-difference estimators according to their inverse variances, is

Q=(ρ/2y_{ρ}σ_{ρ}) [(R₊/T₊)+(R₊/T₊)]⁻¹(T₊^{-1/2}Δp₊+T₋^{-1/2}Δp₋), with

Var(Q)=(sρ/2y_{ρ}²N)[(R₊/T₊)+(R₋/T₋)]₋₁.

An analytical approximation for the repository size required using the combined estimator is

N_{comb}=(sρ/2y_{ρ}²)[(R₊/T₊)+(R₋/T₋)]⁻¹(z_{α}−z_{1-β})²σ_{R}²/σ_{A}².

At the optimal pooling fraction of ρ=0.27, the factor (sρ/2y_{ρ}²) is 2.47. Since the variance of the individual estimators are identical under H₀ and H₁, the repository size for the combined estimator is simply the reciprocal of the sum of the reciprocal repository sizes required for the individual estimators.

4.4 Regression Tests

Regression tests requiring individual genotyping provide a benchmark for the efficiency of tests on pooled DNA. A regression test assesses the significance of the regression coefficient m in the model

Xᵢ=m(pᵢ−p)+εᵢ

where i labels an observation, Xᵢ is an observed phenotype with mean 0 and variance 1, pᵢ is the corresponding observed genotype with mean p, and εᵢ is the residual contribution not explained by the model. For N unrelated individuals, the phenotypic and genotypic variables in the regression test are the individual X₁ and pᵢ values. For N/2 sib-pairs, they are the pair-mean and pair-difference variables X₊ and p₊ for each pair.

The expectation of the regression coefficient m is 0 under H₀ and is

E(m)=σ_{A}/σₚ,

under H₁. The variance of the estimator, assumed identical under both hypotheses with negligible error when σ_{R}² is close to 1, is

Var(m)(s/N)Var(εᵢ)/Var(pᵢ)=(s/N)(T/R)σ_{R}²/σₚ²,

where s=1 for unrelated individuals or 2 for sib-pairs, and T/R=1 for unrelated individuals and T_{±}/R_{±} for pair-mean and pair-difference variables.

The expectation and variance of the test statistic are related to the false-positive rate and power through the equation

[Var(m)]⁻¹ =(z_{α}−z_{1-β})²σ_{R}²/σ_{A}².

Substitution into this equation yields the repository size requirement for the regression test,

N_{reg}=s(T/R)(z_{α}−z_{1-β})²σ_{R}²/σ_{A}².

The combined estimator formed from the pair-mean and pair-difference estimators has a repository size requirement of

N_{regr}=s[R₊/T₊+R₋/T₋]⁻¹(z_{α}−z_{1-β})²σ_{R}²/σ_{A}².

4.5 Computational Methods

Results for required repository sizes were obtained numerically using computations converged to 1×10⁻⁶ [ ]. (ⁱⁱ Press, W H, Teukolsky, S A, Vetterling, W T, and Flannery, B P: Numerical recipes in C, the art of scientific computing, ed 2. Cambridge, UK, Cambridge University Press, 1997.)

Brent's root-finding algorithm was used to determine the threshold values X_{U} and X_{L} for the upper and lower pools for a given pooling design and pooling fraction ρ; Brent's optimization algorithm was then used to find the ρ with maximum power. While the reported results are based on a normal approximation for the allele frequency difference Δp, results were also obtained using the underlying multinomial distribution for the unrelated-population design. The difference between the numerical results for the multinomial and normal distributions was typically less than 1%. The repository size required for the pooling combined estimator was obtained numerically as the reciprocal of the sum of the reciprocal exact sizes required for the pair-mean and pair-difference pooling designs. Using a 750 MHz Pentium III running Linux, the root-finding and minimization for each parameter set required less than 0.01 sec for each design.

To assess the error made by assuming a normal distribution for Δp, we also performed tests in which Δp was calculated exactly according to a multinomial distribution. Results for the required repository size based on the normal distribution were then compared to the repository size based on a multinomial distribution. The two results for N differed by no more than 5% when the number of copies of the minor allele summed over both pools is greater than 60. They differ by approximately 10% when the number of alleles is 10, with the normal distribution underestimating the exact repository size. These differences are not visible on the scale of the figures.

Appendix 4A: Mathematical Details

4A.1 Unrelated Design

The unrelated design considers a population of N unrelated individuals. Upper and lower thresholds X_{U} and X_{L} are defined using

σ=Σ_{G}Φ{−[X_{U}−μ(G)]/σ_{R}}P(G) and

σ=Σ_{G}Φ{[X_{L}−μ(G)]/σ_{R}}P(G),

which may be inverted numerically to find X_{U} and X_{L} as functions of ρ. The probability that an individual selected for a pool has genotype G is denoted σ_{U}(G) for the upper pool and σ_{L}(G) for the lower pool,

θ_{U}(G)=σ⁻¹Φ{−[X_{U}−μ(G)]/σ_{R}}P(G) and

θ_{L}(G)=σ⁻¹Φ{[X_{L}−μ(G)]/σ_{R}}P(G).

The expected allele frequencies under H₁ are

E₁(p_{U})=Σ_{G}θ_{U}(G)p_{G} and

E₁(p_{L})=Σ_{G}θ_{L}(G)p_{G}, with

E₁(Δp)=E(p_{U})−E(p_{L}).

The variance of the test statistic can be obtained from the moments of a multinomial distribution [ ] (ⁱⁱⁱ Beyer W H (ed): CRC Standard Mathematical Tables, ed 27. Boca Raton, CRC Press, 1984.),

σ₀²=2{Σ_{G}P(G)p_{G}²}−2p²=2σₚ² and

σ₁²=Σ_{G}[θ_{U}(G)+θ_{L}(G)]p_{G}²−(p_{U}²+p_{L}²).

Thus, when ρ is specified, the terms in the expression for the repository size N, (z_{α}σ₀−z_{1-β}σ₁)²/ρE₁(Δp)², may all be calculated numerically, and the optimal ρ is obtained by numerical minimization of N as a function of ρ.

An approximate analytical expression for N may be obtained when σ_{R}² is close to 1 by noting that

101 (z−δ)=Φ(z)−yδ,

where y=(2π)^{-1/2}exp{−z²/2}, is correct to lowest order in the small parameter δ. Using μ(G)/σ_{R} as the small parameter δ, the phenotypic thresholds are

X_{U}=−X_{L}=−σ_{R}Φ⁻¹(ρ), and

the expected difference in allele frequency is

E(Δp)=2y_{β}[Σ_{G}P(G)μ(G)p_{G}]/ρσ_{R}=2y_{ρ}σ_{ρ}σ_{A}/ρσ_{R},

where yₚ=(2π)^{-1/2}exp {−[Φ⁻¹(ρ)]²/2)}. To the same order of approximation in μ(G)σ_{R}, both σ₀² and σ₁² may be replaced with 2σₚ². The resulting approximation for the required repository size is

N_{unrelated}=(ρ/2y_{ρ}²)(z_{α}−z_{1-β})²σ_{R}²/σ_{A}².

The minimum occurs at ρ=0.27 and y_{ρ}=0.33.

4A.2 Mahalanobis Design

For the Mahalanobis design, thresholds b_{U} and b_{L} for the radial coordinate are established for the upper and lower pool by solving the following normalization equations:

$$ρ = ( 1 / 2 ) {\sum }_{{G}_{1} , {G}_{2}} P ( {G}_{1} , {G}_{2} ) {\int }_{0}^{π} ⅆ ϕ {\int }_{{b}_{U}}^{∞} ⅆ b b f ( b , ϕ \vert {G}_{1} , {G}_{2} ) and$$

$$ρ = ( 1 / 2 ) {\sum }_{{G}_{1} , {G}_{2}} P ( {G}_{1} , {G}_{2} ) {\int }_{π}^{2 π} ⅆ ϕ {\int }_{{b}_{L}}^{∞} ⅆ b b f ( b , ϕ \vert {G}_{1} , {G}_{2} ) .$$

The factor of (½) arises because only one individual is selected from each sib pair. If the radial coordinate b is larger than the threshold value, the phase angle φ determines which sib is selected for which pool: the sibling with genotype G₁ is selected for the upper pool if 0<φ<π/2 and for the lower pool if π<φπ/2; the sibling with genotype G₂ is selected for the upper pool if π/2<φ<π and for the lower pool if 3π/2<φ<2π. The genotype probabilities θ_{U}(G) and θ_{L}(G) for the upper and lower pools may be written

$${θ}_{U} ( G ) = {ρ}^{- 1} {Σ}_{{G}^{′}} P ( G , {G}^{′} ) {\int }_{0}^{π / 2} ⅆ ϕ {\int }_{{b}_{U}}^{∞} ⅆ b b f ( b , ϕ \vert G , {G}^{′} ) and$$

$${θ}_{L} ( G ) = {ρ}^{- 1} {Σ}_{{G}^{′}} P ( G , {G}^{′} ) {\int }_{π}^{3 π / 2} ⅆ ϕ {\int }_{{b}_{L}}^{∞} ⅆ b b f ( b , ϕ \vert G , {G}^{′} ) ,$$

where symmetry between siblings has allowed the change in integration limits for φ to consider only the regions where sibling 1 is selected. Once ρ is specified, the thresholds for b may be obtained numerically, and E₁(Δp) may be obtained from θ_{U} and θ_{L}. Numerical results for the required repository size may then be obtained as outlined above for the unrelated design.

An analytic approximation for the repository size requirement may be obtained by noting that

f(b,φ|G₁,G₂)=(2π)⁻¹[1+(bν₊)cosφ+(bν₋)sinφ]exp(−b²/2)

to lowest order in the gene effect μ(G). The normalization condition leads to the equation

ρ=(¼)exp(−b_{ρ}₂/2),

with b_{U}=b_{L}=b_{ρ} defined in terms of the pooling fraction ρ. The genotype frequencies in the upper and lower pools are

θ_{U,L}(G)=P(G)±Σ_{G′}P(G,G′)(ν₊+ν₋)[(2b_{ρ}/π)+Φ(−b_{ρ})/ρ(2π)^{1/2}],

where the upper pool has the + sign and the lower pool the − sign. The expected allele frequencies in the upper and lower pools are

E(p_{U,L})=p±[(2b_{ρ}/T)+Φ(−b_{ρ})/ρ(2π)^{1/2}][R₊/T₊^{1/2}+R₊/T₋^{1/2}]σₚσ_{A}/σ_{R},

where the upper pool has the positive deviation from p and the lower pool the negative deviation. These results are derived using the identities

$$\begin{matrix}{\sum }_{{G}_{1} , {G}_{2}} P ( {G}_{1} , {G}_{2} ) μ ( {G}_{1} ) {p}_{{G}_{1}} = ( 1 / r ) {\sum }_{{G}_{1} , {G}_{2}} P ( {G}_{1} , {G}_{2} ) μ ( {G}_{1} ) {p}_{{G}_{2}} \\ = {σ}_{A} {σ}_{p}\end{matrix}$$

where r is the genotypic correlation (0.5 for full-sibs). Since θ_{U}(G)+θ_{L}(G) is 2P(G), the variance term σ₁² is equal to σ₀², and both are equal to 2σₚ² because all the pooled individuals are unrelated. The approximate expression for the number of individuals required for the Mahalanobis design is

N_{Mahalanobis}=(2ρ)⁻¹[(2b_{ρ}/π)+Φ(−b_{ρ})/ρ(2π)_{1/2}]₂[R₊/T₊^{1/2}+R₋/T₋^{1/2}]⁻²(z_{α}−z_{1-β})²σ_{R}²/σ_{A}².

The minimum occurs at ρ=0.188.

4A.3 Pair-Mean Design

The fraction ρ of the total population selected according to pair-mean pooling is defined in terms of the upper threshold X_{U} and the lower threshold X_{L} as

$$\begin{matrix}ρ = {\sum }_{{G}_{1} , {G}_{2}} P ( {G}_{1} , {G}_{2} ) Φ \{ - [ {X}_{U} - {μ}_{+} ( {G}_{1} , {G}_{2} ) ] / {σ}_{+} \} \\ = {\sum }_{{G}_{1} , {G}_{2}} P ( {G}_{1} , {G}_{2} ) Φ \{ [ {X}_{L} - {μ}_{+} ( {G}_{1} , {G}_{2} ) ] / {σ}_{+} \} .\end{matrix}$$

The genotype distribution describing the individuals selected for each pool follows a multinomial distribution based on sib-pair genotypes rather than individual genotypes, such that

$$1 = {\sum }_{{G}_{1} , {G}_{2}} {θ}_{U} ( {G}_{1} , {G}_{2} ) = {\sum }_{{G}_{1} , {G}_{2}} {θ}_{L} ( {G}_{1} , {G}_{2} ) ,$$

with

θ_{U}(G₁,G₂)=ρ⁻¹Φ{−[X_{U}−μ(G)/σ_{R}}P(G₁,G₂) and

θ_{L}(G₁,G₂)=ρ⁻¹Φ{[X_{L}−μ(G)/σ_{R}}P(G₁,G₂).

The expected allele frequencies under H₁ are

$$\begin{matrix}{E}_{1} ( {p}_{U} ) = {\sum }_{{G}_{1} , {G}_{2}} {θ}_{U} ( {G}_{1} , {G}_{2} ) {p}_{+} ( {G}_{1} , {G}_{2} ) and \\ {E}_{1} ( {p}_{L} ) = {\sum }_{{G}_{1} , {G}_{2}} {θ}_{L} ( {G}_{1} , {G}_{2} ) {p}_{+} ( {G}_{1} , {G}_{2} ) , with\end{matrix}$$

E₁(Δp)=E(p_{U})−E(p_{L})

and p₊(G₁,G₂) is the pair-mean allele frequency as defined previously. The terms giving the variance of the test statistic under H₀ and H₁ are

$$\begin{matrix}{σ}_{0}^{2} = 2 s \{ {\sum }_{{G}_{1} , {G}_{2}} {P ( {G}_{1} , {G}_{2} ) [ {p}_{+} ( {G}_{1} , {G}_{2} ) ]}^{2} \} - 2 {sp}^{2} \\ = 2 {sR}_{+} {σ}_{p}^{2} \\ = 3 {σ}_{p}^{2} and \\ {σ}_{1}^{2} = s \{ {\sum }_{{G}_{1} , {G}_{2}} {[ {θ}_{U} ( {G}_{1} , {G}_{2} ) + {θ}_{L} ( {G}_{1} , {G}_{2} ) ] [ {p}_{+} ( {G}_{1} , {G}_{2} ) ]}^{2} \} - \\ s ( {p}_{U}^{2} + {p}_{L}^{2} ) .\end{matrix}$$

The factor s=2 accounts for the family structure, as n/s rather than n measurements of p₊ are used to determine the allele frequency of each pool. The variance under the null hypothesis may be derived directly from the sib-pair genotype frequencies, or more simply by noting that the variance of the mean allele frequency for a sib-pair is R₊σₚ², which is (¾) of the variance σₚ² for an individual. Taking the mean of n/2 such terms reduces the variance for each pool by n/2. The total variance is obtained by multiplying by 2 for the number of pools, yielding 3σₚ². Given σ, the pooling thresholds are obtained numerically, then used to calculate E₁(Δp) and σ₁², yielding a numerical result for the repository size N as a function of ρ.

An analytical approximation follows the same derivation used for the unrelated design, except that individual genotypes are replaced by sib-pair genotypes, and individual phenotypes, phenotype offsets, and allele frequencies are replaced by their pair-mean analogs. The upper and lower pooling thresholds are

X_{U}=−X_{L}=−σ₊Φ⁻¹(ρ),

and the allele frequency difference between pools is

$$\begin{matrix}E ( Δ p ) = 2 {y}_{ρ} [ {\sum }_{{G}_{1} , {G}_{2}} P ( {G}_{1} , {G}_{2} ) {μ}_{+} ( {G}_{1} , {G}_{2} ) {p}_{+} ( {G}_{1} , {G}_{2} ) ] / {ρσ}_{+} \\ = ( 2 {y}_{ρ} / ρ ) ( {R}_{+} / {T}_{+}^{1 / 2} ) {σ}_{p} {σ}_{A} / {σ}_{R} ,\end{matrix}$$

where y_{ρ} is the height of the standard normal density at Φ⁻¹(ρ) as before. The contributions of the corresponding low-order terms in σ₁² cancel, and the variance of Δp is the same under both hypotheses. The repository size required by the pair-mean design is

Nₚₐᵢᵣ₋ₘₑₐₙ=(sρ/2y_{ρ}²)(T₊/R₊)(z_{α}−z_{1-β})²σ_{R}²/σ_{A}².

4A.4 Pair-Difference Design

Under the pair-difference design, a sib pair is selected if the pair-difference X₋ is larger in magnitude than a threshold X_{T},

$$\begin{matrix}2 ρ = {\sum }_{{G}_{1} , {G}_{2}} P ( {G}_{1} , {G}_{2} ) Φ \{ [ {μ}_{-} ( {G}_{1} , {G}_{2} ) - {X}_{T} ] / {σ}_{-} \} + \\ {\sum }_{{G}_{1} , {G}_{2}} P ( {G}_{1} , {G}_{2} ) Φ \{ - [ {μ}_{-} ( {G}_{1} , {G}_{2} ) + {X}_{T} ] / {σ}_{-} \} .\end{matrix}$$

In the first term, sibling 1 has the higher phenotype and is selected for the upper pool, and sibling 2 is selected for the lower pool. In the second term, the roles of the siblings are reversed. Multinomial distributions are defined as θ_{U}(G₁,G₂), the genotype probabilities for sib pairs in which sibling 1 enters the upper pool, and θ_{L}(G₁,G₂), when sibling 1 enters the lower pool. For selected pairs,

$$1 = {\sum }_{{G}_{1} , {G}_{2}} \{ {θ}_{U} ( {G}_{1} , {G}_{2} ) + {θ}_{L} ( {G}_{1} , {G}_{2} ) \} .$$

This normalization implies that

θ_{U}(G₁,G₂)=(2ρ)⁻¹P(G₁,G₂) Φ{[μ(G₁,G₂)−X_{T}]/σ₋} and

θ_{L}(G₁,G₂)=(2ρ)⁻¹P(G₁,G₂) Φ{−[μ(G₁,G₂)+X_{T}]/σ₋}.

Due to symmetry, θ_{U}(G₁,G₂) and θ_{L}(G₂,G₁) are identical. The expected allele frequency difference between pools is

$$\begin{matrix}{E}_{1} ( Δ p ) = {\sum }_{{G}_{1} , {G}_{2}} 2 {θ}_{U} ( {G}_{1} , {G}_{2} ) {p}_{-} ( {G}_{1} , {G}_{2} ) - \\ {\sum }_{{G}_{1} , {G}_{2}} 2 {θ}_{L} ( {G}_{1} , {G}_{2} ) {p}_{-} ( {G}_{1} , {G}_{2} ) ;\end{matrix}$$

by symmetry, each term contributes E(Δp)/2. To calculate the variance of Δp, it is important to note that the normalization of θ_{U} and θ_{L} to ½0 implies that the probabilities for a multinomial distribution are 2 θ_{U} and 2 θ_{L}, with both θ_{U} and θ_{L} equal to P(G], G₂)/2 under the null hypothesis. The terms giving the variance under the null hypothesis and the alternative hypothesis are

$${σ}_{0}^{2} = 2 s {\sum }_{{G}_{1} , {G}_{2}} P ( {G}_{1} , {G}_{2} ) {p}_{-}^{2} = 2 {sR}_{-} {σ}_{p}^{2} = {σ}_{p}^{2} and$$

$${σ}_{1}^{2} = 2 {\sum }_{{G}_{1} , {G}_{2}} [ 2 {θ}_{U} ( {G}_{1} , {G}_{2} ) + 2 {θ}_{L} ( {G}_{1} , {G}_{2} ) ] {p}_{-}^{2} - {E ( Δ p )}^{2} .$$

The value of σ₀² under the null hypothesis may be obtained more simply by noting that the allele frequency difference between two siblings has variance σₚ², and the measured allele frequency difference is the mean of n such terms.

The repository size required to detect association may be determined exactly by numeric calculation of the threshold value X_{T} as a function of the pooling fraction ρ. This value is then used to determine E(Δp), σ⁰², and σ₁².

An analytic expression accurate when σ_{R}² is close to 1 may be derived using the same technique as for the previous pooling designs. The analytic estimate for the threshold value is

X_{T}=−σ₋Φ⁻¹(β)

and the allele frequency difference is

$$\begin{matrix}E ( Δ p ) = 2 {y}_{ρ} [ {\sum }_{{G}_{1} , {G}_{2}} P ( {G}_{1} , {G}_{2} ) {μ}_{-} ( {G}_{1} , {G}_{2} ) {p}_{-} ( {G}_{1} , {G}_{2} ) ] / p {σ}_{-} \\ = ( 2 {y}_{ρ} / ρ ) ( {R}_{-} / {T}_{-}^{1 / 2} ) {σ}_{p} {σ}_{A} / {σ}_{R}\end{matrix}$$

where yₚ is the height of the standard normal density at Φ⁻¹(ρ). The variance term σ₁² equals σ₀² to this order of approximation, and the repository size required by the pair-difference design is

N_{parr-diff}=(sρ/2y_{ρ}²)(T₋/R₋)(z_{α}−z_{1-β})²σ_{R}²/σ_{A}².

### EXAMPLE 4.1

### Comparisons with individual genotyping

When the effect of a QTL is small and the residual variance σ_{R}² is close to 1, the analytic expressions for repository size requirements are exact. In this limit, we begin by comparing the efficiency of pooled DNA designs relative to individual genotyping.

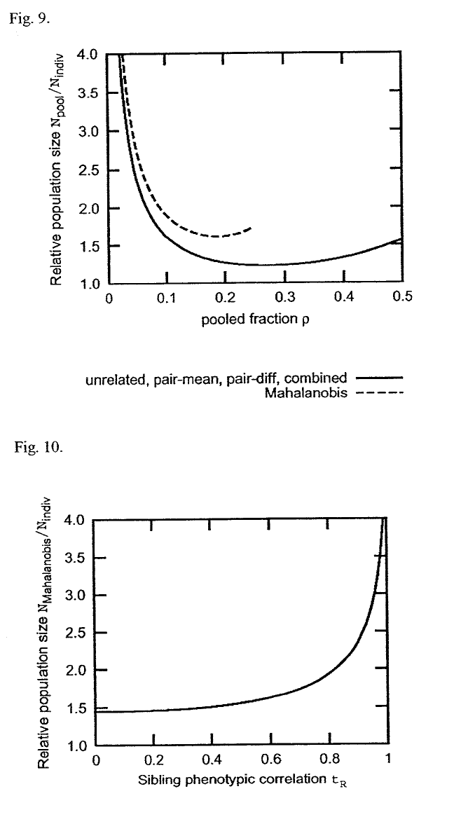

The repository size requirements of pooled DNA methods are shown in FIG. 9 relative to the corresponding regression tests for the same family structure. Methods plotted are the unrelated, pair-mean, pair-difference, and combined designs, as well as the Mahalanobis design. Except for the Mahalanobis design, the ratio of repository size requirements is independent of all model parameters except for the fraction ρ of individuals whose DNA is pooled. Furthermore, the ratio is independent of family structure for these matched comparisons. The optimal pooling fraction is ρ=0.27. The curves are flat near the minimum, indicating that pooling fractions close to the optimum give near-optimal results. Repository sizes must be increased by 1.24× to attain the same power as would have been achieved with N individual genotypes.

The repository size required for the Mahalanobis design is shown relative to that required for the combined regression test. This ratio depends on the residual phenotypic correlation t_{R}between siblings, and a typical value t_{R}=0.6 has been selected for illustrative purposes. The minimum at 0.188 is independent of t_{R}, and the repository must be 1.55× larger than that for a genotyping study.

In FIG. 10, the performance of the Mahalanobis design relative to the combined regression test for individual genotypes is shown as a function of the residual sibling phenotypic correlation t_{R}, with the optimal fraction 0.188 used to construct the upper and lower pools. The ratio of repository sizes is roughly 1.5 until the phenotypic correlation rises above 0.6, at which point the repository size requirements for the Mahalanobis design begin to rise more steeply.

### EXAMPLE 4.2

### Comparisons Between Unrelated and Sib-Pair Populations

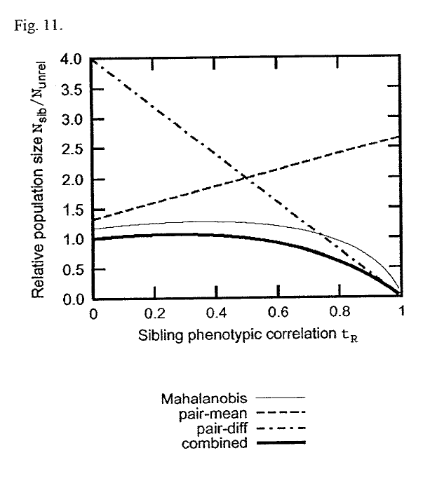

In FIG. 11, the repository size requirements for association tests using DNA pooled from sib pairs are shown as a function of the residual sibling phenotypic correlation t_{R}, relative to the repository size required for a test of DNA pooled from unrelated individuals. Ratios larger than 1 indicate that the population of N unrelated individuals is more powerful than a population of N/2 sib pairs, while ratios smaller than 1 indicate that the sib-pair population is more powerful. These ratios are derived from the analytical approximations derived for complex traits.

For designs using only 2 pools, a population of unrelated individuals is more powerful than a population of sib pairs except for large values of the sibling phenotypic correlation, t_{R}>0.75, at which point the Mahalanobis and pair-difference designs become more powerful. Below this phenotypic correlation, the Mahalanobis design is substantially more powerful than the other sib-pair tests; above this correlation, the pair-difference design is only slightly more powerful than the Mahalanobis design.

The slope of the pair-difference repository size requirement is 3× larger than the slope of the pair-mean population requirement. Thus, relative to the pair-mean design, the pair-difference design decreases in power rapidly as t_{R} falls below 0.5 and increases in power rapidly as t_{R}rises above 0.5.

The combined 4 pool test using pair-mean and pair-difference pools is uniformly the most powerful sib-pair design for all values of t_{R}. Its worst-case performance relative to an unrelated population occurs when t_{R} is (3^{1/2}+1)/(3^{1/2}−1), or 0.2679, where it requires a population 7% larger. The unrelated and sib-pair tests require the same repository size when the phenotypic correlation is 0.5, and the sib-pair test becomes much more powerful For equal repository sizes for larger values of t_{R}.

### EXAMPLE 4.3

### Sensitivity to QTL Effect Size, Allele Frequency, and Inheritance Mode

According to the analytic theory, the necessary size of the study population for pooling tests is inversely proportional to the additive variance contributed by the QTL relative to the residual phenotypic variance, σ_{A}²/σ_{R}², and independent of any remaining parameters of the genetic model. Here we provide exact numerical results to assess the region of validity for the analytical approximations. For these numerical results, the type I error rate α is 5×10⁻⁸ and the type II error rate β is 0.2 to provide adequate power and an acceptable number of false-positives for a whole-genome scan. For consistency in FIGS. 4-6, the unrelated-population design is a dotted line, Mahalanobis is a thin line, pair-mean is dashed, pair-difference is dot-dashed, and the combined estimator sib-combined is a thick line.

A single representative value for the sibling phenotypic correlation t_{R} was selected for these tests. This correlation is equal to half the genetic heritability plus the shared enviromnental contribution to the total variance of a complex trait. For cancer, heritability has been estimated at 20% and environmental factors at 7% (Verkasalo et al., 1999); for systolic and diastolic blood pressure, heritabilities are estimated at 10% to 50% and environmental factors at 20% to 40% [,] (Iselius et a., 1983; Perusse et al., 1989); heritability for cholesterol level is estimated at 70% to 90% (Austin et al., 1987) and environmental factors for serum lipids are estimated 15% [] (^{viii} Heller D A, de Faire U, Pedersen N L, Dahlen G, McClearn G E: Genetic and environmental influences on serum lipid levels in twins. N Engl J Med 1993; 328;: 1150-6). Additional heritability estimates are 20% to 40% for Type 2 diabetes mellitus (NIDDM) l Watanabe R M, Valle T, Hauser E R, Ghosh S, Eriksson J, Kohtamaki K, Ehnholm C Ehnholm C, Tuomilehto J, Collins F S, Bergman R N, Boehnke M: Familiality of quantitative metabolic traits in Finnish families with non-insulin-dependent diabetes mellitus. Finland-United States Investigation of NIDDM Genetics (FUSION) Study investigators. Hum Hered 1999; 49: 159-168] and 50% for pulmonary function [^{x} Wilk J B, Djousse L, Arnett D K, Rich S S, Province M A, Hunt S C, Crapo R O, Higgins M, Myers R H: Evidence for major genes influencing pulmonary function in the NHLBI family heart study. Genet Epidemiol 2000; 19: 81-94]. These values suggest a range of 0.25 to 0.75 for t_{R}; we selected t_{R}=0.6. Choosing a different value of t_{R} changes the relative power of different pooling designs, as shown in FIG. 11, but does not alter any conclusions regarding the validity of the analytic theory.

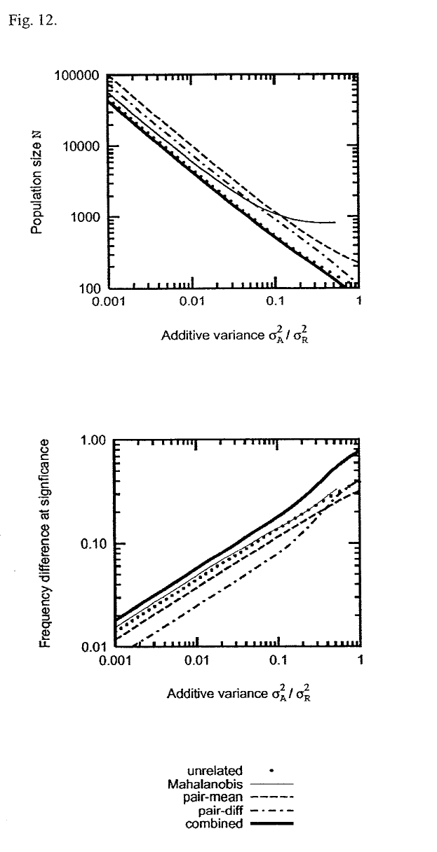

In FIG. 12, the ratio σ_{A}²/σ_{R}² is varied over 3 orders of magnitude. The QTL has purely additive inheritance and the minor allele frequency is 0.1. The pooling fraction has been optimized numerically, and linearity in the log-log plot demonstrates validity of the analytic results. Inspection of the results shows that agreement extends almost to σ_{A}²/σ_{R}²=1, where the QTL is responsible for half the phenotypic variance, for all the designs except Mahalanobis. The Mahalanobis design is less powerful than predicted by analytic theory for σ_{A}²/σ_{R}²>0.05. This level of additive variance marks the onset of a major gene effect: carriers of the minor allele separating into a clearly resolved affected population, and the association may be identified by traditional family-based linkage analysis.

The allele frequency difference at the significance threshold, z_{α}σ₀/n^{1/2}, is shown in FIG. 12B for the same set of parameters. For the combined design, there are actually two frequency differences, one for the pair-mean pools and another for the pair-difference pools. Only the difference for the pair-difference pools is shown. As the QTL contribution becomes smaller, allele frequency differences must be measured with greater precision. While raw frequency differences of 10% are significant for a major gene σ_{A}²/σ_{R}²˜0.1), raw frequencies differences of 3% must be measured with little error to achieve maximum power for a complex trait with σ_{A}²/R²˜0.01.

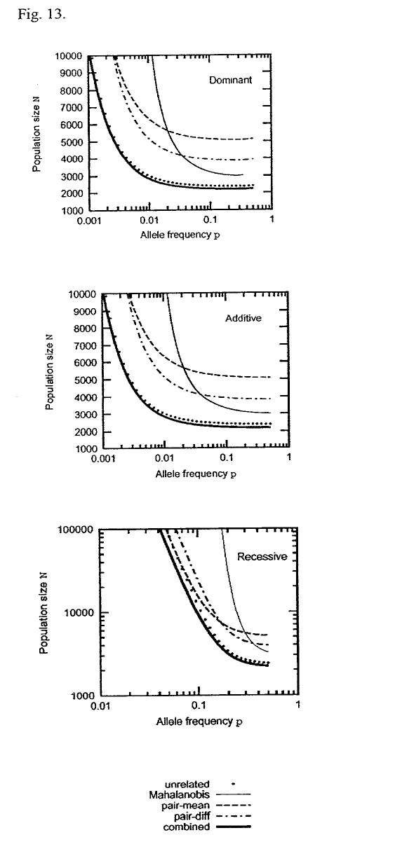

The sensitivity of the results to both the allele frequency p and the inheritance mode are shown in FIGS. 5 and 6. In both of these figures, the pooling fractions are fixed at the limiting values 0.27 for the unrelated-population, pair-mean, pair-difference, and sib-combined designs and at 0.188 for the Mahalanobis design, as would be presumably be done if DNA is pooled once then used repeatedly in a genome-wide screen of markers. In FIG. 13, the allele frequency is varied for a phenotype with dominant inheritance (FIG. 13A), additive inheritance (FIG. 13B), and recessive inheritance (FIG. 13C) of the minor allele. The QTL contribution σ_{A}²/σ_{R}² is held fixed at 0.02 for these comparisons. The figures are shown only for the region p<0.5 on a log scale to highlight the behavior for small values of p; additive alleles are symmetric al)out p=0.5, while dominant major alleles are equivalent to recessive minor alleles and vice versa. It is important to note that the displacements a and d are increased to compensate for a smaller allele frequency p in order to keep σ_{A}² constant and ensure that the limiting behavior for a QTL with small effect is a horizontal line. If the displacements had been held constant, then σ_{A}² would decrease linearly with p and the required repository size would increase as 1/p.

The repository size is rather insensitive to allele frequency for p>0.01 for dominant and additive inheritance, and for p>0.2 for recessive inheritance, for all but the Mahalanobis design, indicating that the analytic theory is valid in these regions. The repository size required to detect association increases rapidly as the allele frequency decreases below these limits. The Mahalanobis design is more sensitive to the allele frequency than the other designs, losing power rapidly as the allele frequency falls below 0.1 for dominant and additive inheritance and 0.2 for recessive inheritance.

The allele frequency at which the analytic theory loses accuracy may be estimated by noting that the perturbation parameters used to derive the theory are the terms μ(G)/σ_{R}. As the magnitude of these terms approaches 1, or equivalently when the displacements a or d become close to 1, the perturbation theory becomes less reliable. This occurs for p=σ_{A}²/8 under dominant inheritance, σ_{A}²/2 under additive inheritance, and σ_{A}^{2/3}/2 for recessive inheritance. In FIG. 13, these values are 0.0025, 0.01, and 0.14, and accurately identify the elbows of the repository size curves.

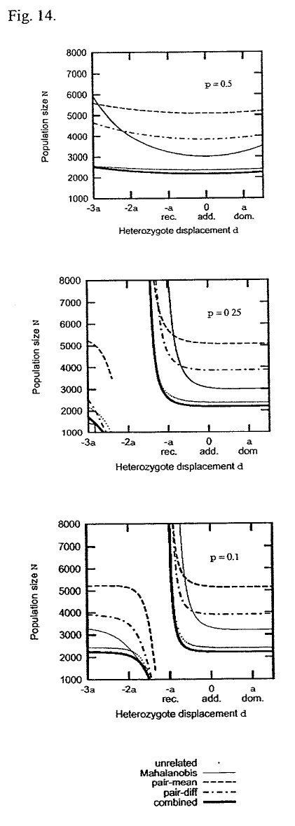

In FIG. 14, the mode of inheritance is varied while the allele frequency is held fixed at one of three values, p=0.5 (FIG. 14A), 0.25 (FIG. 14B), or 0.1 (FIG. 14C). When p=0.5, the inheritance mode has virtually no effect on the repository size required to detect association. The Mahalanobis design is an exception, with increasing requirements only for strong over-dominance. For p<0.5, the additive variance necessarily vanishes at d=a/(2p−1); when d is close to this value, the population requirements increase dramatically. For p=0.25, this occurs at d=−2a. Above this critical value of d, excess A₁A₁ homozygotes are detected in the upper pool; below the critical value, excess A₁A₂ heterozygotes are detected in the lower pool. Although Δp is negative in this region and therefore not significant under a one-sided test of allele A₁, a two-tailed test would yield a significant result. The repository size requirements are substantially smaller than predicted by analytic theory for this region of strongly over-dominant major alleles.

In the bottom panel, FIG. 14C, the allele frequency is p=0.1 and the critical value of d is −1.125 a. The region of increased population requirements is narrower than in FIG. 14B, and becomes narrower still when p is further reduced, but the general behavior is the same.

### EXAMPLE 4.4

### Dependence on Type I and Type II Error Rates

We have also investigated the sensitivity of the exact numerical results to specified rates of type I and type II error. In the analytical approximations, this behavior is described entirely by the term (z_{α}−z_{1-β})², and the optimal pooling fractions are independent of α and β. Comparison with numerical results indicate that the analytical theory is accurate, with no differences seen on the scale of the figures previously presented (results not shown). Using the (z_{α}−z_{1-β})² scaling and specifying a fixed power of 0.8 (z_{1=β}=−0.84), for example, a whole-genome scan with α=5×10 ⁻⁸ (z_{α}=5.33) requires 1.7× more individuals than a test of 1000 candidate markers with α=5×10⁻⁵ (z_{α}=3.89) and 6.2× more individuals than a test of a single marker with α=0.05 (z_{α}=1.64).

### EXAMPLE 4.5

### Tests in the Presence of Population Stratification

A marker may show spurious association to a phenotype in the presence of a stratified population. We consider a simple model for stratification in which a population contains at least one sub-population having a mean marker frequency and a mean phenotypic value that both deviate from their respective means in the total population. In individual genotyping, within-family tests such as the transmission disequilibrium test are known to be robust to this type of stratification. Between-family tests, however, may identify spurious associations or miss true associations due to stratification effects.

Tests of pooled DNA in which family members are balanced between pools, such as the pair-difference design, are analogous to within-family tests. The value of σ_{A}/σ_{R} estimated from this test is robust to stratification effects. The remaining designs, in particular the pair-mean design, do not balance family members and are subject to stratification. A suitable test for the presence of stratification, therefore, is to compare the value of σ_{A}/σ_{R} estimated separately from the pair-difference and pair-mean pools with the combined estimator in the form of a X² test,

X²={[Q₊−Q]²/[sρ/2y_{ρ}²N][T₊/R₊]}+{[Q₋−Q]²/[sρ/2y_{ρ}²N][T₋/R₋]},

with one degree of freedom. This stratification estimator may also be expressed as

X²=[Q₊−Q₋]²/{[sρ/2y_{ρ}²N][T₊/R₊+T₋/R₋]}.

A significant finding for this test, for example at the 0.05 level, indicates that stratification is present and that tests other than the pair-difference test may yield spurious results.

### EXAMPLE 4.6

### Allele Frequency Measurement Error

The preceding analysis has assumed that allele frequency measurement errors are negligible. Allele frequencies measured by most technologies, including PCR amplification [^{xi} Shaw S H, Carrasquillo M M, Kashuk C, Puffenberger E G, Chakravarti A: Allele frequency distributions in pooled DNA samples: applications to mapping complex disease genes. Gen Res 1998; 8; 111-123], kinetic PCR [^{xll} Germer S, Holland M J, Higuchi R. High-throughput SNIP allele-frequency determination in pooled DNA samples by kinetic PCR. Gen Res 2000; 10; 258-266], denaturing high performance liquid chromatography [^{xlii} Hoogendoorn B, Norton N, Kirov G, Williams N, Hamshere M L, Spurlock G, Austin J, Stephens M K, Buckland P R, Owen M J, O'Donovan M C: Cheap, accurate and rapid allele frequency estimation of single nucleotide polymorphisms by primer extension and DHPLC in DNA pools. Hum Gen 2000; 107; 488-493], single-strand conformation polymorphism [^{XIV} Sasaki T, Tahira T, Suzuki A, Higasa K, Kukita Y, Baba S, Hayashi K: Precise estimation of allele frequencies of single-nucleotide polymorphisms by a quantitative SSCP analysis of pooled DNA. Am J Hum Gen 2001; 68; 214-218], pyrophosphate sequencing [^{XV} Alderborn A, Kristofferson A, Hammerling U: Determination of single-nucleotide polymorphisms by real-time pyrophosphate DNA sequencing. Genome Res 2000; 10; 1249-1258], and mass spectrometry [^{XVI} Buetow K H, Edmonson M, MacDonald R, Clifford R, Yip P, Kelley J, Little D P, Strausberg R, Koester H, Cantor C R, Braun A: High-throughput development and characterization of a genomewide collection of gene-based single nucleotide polymorphism markers by chip-based matrix-assisted laser desorption/ionization time-of-flight mass spectrometry. Proc Nat Acad Sci USA 2001; 98; 581-584], are typically reported with standard errors in the range of 0.01 to 0.02. Assuming a measurement error of 0.01 for p_{U} and p_{L}, the resulting error in the population mean estimated as p=(p_{U}+p_{L})/2 is 0.007. The measurement error in p affects the calculated repository size N primarily through the terms and σ₁², which are proportional to p(1−p). The relative error in N is proportional to 0.007/p, less than 10% for minor alleles with frequency greater than 0.07.

The measurement error in Δp, however, has a more deleterious affect on the test power. Again assuming a measurement error of 0.01 for each pool, the measurement error for Δp is √2 larger, approximately 0.014. This error can eventually become larger than the sampling error σ₀²/n for large values of n. In this case, the critical value of Δp depends on the measurement error, not the sampling error. For example, the magnitude of Δp for a two-sided test with significance at the 0.01 level and power 0.95 is (z_{0.005}−z_{0.95})×0.014, or 0.059 using z_{0.0005}=2.58 and z_{0.95}=−1.64.

The allele frequency measurement error also sets a lower limit for the effect size that can be detected with a pooled test. For example, using the analytical approximation for Δp for pair-mean pools derived in the Appendix,

E₁(Δp)=(2y_{ρ}/ρ)(R₊/T₊^{1/2})σₚσ_{A}/σ_{R}≈2.6×(1+t_{R})^{-1/2}p(1−p)|a−(2p−1)d|>0.059,

where the optimized pooling fraction ρ=0.27 is used and the residual variance σ_{R}²is approximated as 1. For a typical phenotypic correlation between sibs, t_{R} is 0.5, and the effect size that can be detected is

|a−(2p−1)d|>0.028/p(1−p).

For additive inheritance and allele frequency of 0.5, the threshold phenotypic displacement a is 0.11 and the corresponding additive variance is 0.0063. If the minor allele frequency is 0.1, the threshold displacement a is 0.31 and the corresponding additive variance is 0.017.

In the presence of population stratification, the pair-mean pools may give spurious results and pair-difference pools are preferred. Using the expectation for Δp derived in the Appendix for pair-difference pools, we require that

E₁(Δp)=(2y_{ρ}/ρ)(R₋/T₋^{1/2})σₚσ_{A}/σ_{R}≈0.86×(1+t_{R})^{-1/2}p(1−p)|a−(2p−1)d|>0.059,

where ρ=0.27 and σ_{R}²≈1 as before. For a typical phenotypic correlation between sibs, t_{R}=0.5, the effect size that can be detected is

|a−(2p−1)d|>0.049/p(1−p).

For additive inheritance and an allele frequency of 0.5, the critical displacement is 0.20 and the additive variance is 0.02. For a rare minor allele, p=0.1, and additive inheritance, the critical displacement is 0.54, corresponding to an additive variance of 0.05.

5. Model 3

In this model techniques similar to those described in Models 1 and 2 are applied to provide optimized selection criteria for association studies of pooled DNA using the allele frequency difference between pools as a test statistic. It is assumed that samples are drawn from pre-existing population-level DNA repository collected from individuals unselected for any particular phenotype, and that each individual has been measured for a particular phenotype of interest; the goal is to select pools to maximize the power of the test.

Assuming no experimental error in allele frequency measurements on pooled DNA, we determine the selection thresholds that maximize the power to detect association as a function of the frequency, phenotypic displacement, and inheritance mode of a functional polymorphism. The genetic parameters are also described in terms of a genotype relative risk model. Power calculations are then used to derive the repository size required to detect association at specified false-positive and false-negative rates. These calculations are performed at three decreasing levels of accuracy: exact numerical calculations using the true multinomial distribution of the test statistic; numerical calculations based on an approximate normal distribution of the test statistic; and analytical approximations accurate for complex traits where the polymorphism has a small effect on the phenotype.

Results are depicted in terms of the repository sizes required for three types of experimental designs for detecting association with a quantitative phenotype: first, a pooled DNA test using a conventional affected/unaffected classification; second, a pooled DNA test of extreme individuals using optimized selection thresholds; third, individual genotyping of the entire population. We conclude with a discussion of the reduction in power of pooled DNA tests due to experimental measurement error and with suggestions for effective use of pooled DNA tests in practice.

5.1 Computational Methods

The calculation of optimized selection thresholds begins with a model for the genotype-dependent distribution of phenotypic values. A quantitative phenotype, denoted X, is standardized to have unit variance and zero mean. The phenotype is hypothesized to be affected by alleles A₁ and A₂, with frequencies p and 1−p respectively, at a particular QTL. The population frequencies P(G) for genotypes G=A₁A₁, A₁A₂, and A₂A₂ are assumed to obey Hardy-Weinberg equilibrium. Using standard notation for a variance components model, the effect μ_{G} of genotype G on phenotype X is a, d and −a , for genotypes A₁A₁, A₁A₂, and A₂A₂ respectively. These displacements are each offset by subtracting (2p−1)a+2p(1−p)d to preserve the overall phenotype mean of zero.

The inheritance mode of the QTL is represented by the displacement d of the heterozygote, for example purely recessive (d=−a), additive (d=0), or dominant (d=+a) inheritance, The inheritance mode partitions the phenotypic variance due to the QTL into the additive variance σ_{A}² and the dominance variance σ_{D}², with

σ_{A}²+σ_{D}²=2p(1−p)[a−d(2p−1)]²+4p²(1−p)²d².

This partitioning is important because, as will be seen below, pooled tests are sensitive primarily to the additive component of variance. Note that the additive component may be large even when the inheritance is purely dominant or recessive. The contributions to the phenotype from remaining genetic and environmental factors are assumed to follow a normal distribution with residual variance σ_{R}²,

σ_{A}²=1−(σ_{A}²+σ_{D}²).

The genotype-dependent phenotype distributions for each genotype are

P(X|G)=(2πσ_{R}²)^{-1/2}exp[−(X−μ_{G})²/2σ_{R}²],

normal distributions centered at μ^{G} with width σ_{R}. The overall phenotype distribution is the weighted sum of the distributions from each genotype,

P(X)=Σ_{G}P(X|G)P(G).

For a complex trait in which the QTL makes a small contribution, the three underlying distributions may be unresolved in the observed P(X).

This variance components model may be connected to an equivalent affected/unaffected genotype relative risk model by specifying a threshold phenotypic value X_{T} that classifies individuals as affected (X>X_{T}) or unaffected (X<X_{T}). The proportion r of the total population that is affected is the overall risk or disease prevalence; the probability that an individual with genotype G is affected, divided by the corresponding probability for an individual with genotype A₂A₂, is the genotype relative risk.

In the tests of pooled DNA considered here, a sample repository of total size N serves as the source of DNA to be selected for one of two pools; not every individual need be selected. The test statistic is the difference in the frequency that a particular allele, here always assumed to be A₁, occurs in the two pools. For a quantitative phenotype, it is natural to specify an upper threshold X_{U} and a lower threshold X_{L} as the selection criteria. Individuals with phenotypic values above X_{U} are selected for the upper pool; individuals with phenotypic values below X_{L}are selected for the lower pool; and individuals with phenotypic values between X_{L} and X_{U} are not pooled at all. The number of individuals selected for each pool is ρN. The fraction ρ expressed in terms of X_{U} is

ρ=Σ_{G}Φ[−(X_{U}−μ_{G})/σ_{R}]P(G),

which is solved numerically to determine X_{U}. The genotypes of individuals selected by X>X_{U}, follow a multinomial distribution; the probability θ_{U}(G) that an individual selected for this pool has genotype G is Φ[−X_{U}−μ_{G})/σ_{R}]P(G)/ρ. A multinomial distribution is defined similarly for the lower pool,

1=Σ_{G}θ_{L}(G)=ρ⁻¹Σ_{G}Φ[(X_{L}−μ_{G})/σ_{R}]P(G),

using the lower threshold X_{L},

A pooling design based on an affected/unaffected classification is similar: affected individuals are selected for the upper pool; an equivalent number of suitably matched unaffected individuals are selected for the lower pool. The selection thresholds X_{U} and X_{L} are identical to the classification threshold X_{T}. The relative risk for genotype G, expressed in terms of the pooling threshold, is [θ_{U}(G)/P(G)]/ [θ_{U}(A₂A₂)/P(A₂A₂)].

The repository size N required to detect association between genotype G and either the quantitative phenotype X or the affected/unaffected classification depends on the desired type I error rate α and type II error rate β, the chosen test statistic, and the experimental design, as well as on the underlying genetic model. For a one-sided test of a single marker, α=1−Φ(z_{α}) and 1−β=Φ(−z_{-β}), where Φ(z) is the cumulative probability distribution for standard normal deviate z. For a genome scan, the values α=5×10⁻⁸ (z_{α}=5.33) and 1−β=0.8 (z_{1-β}=−0.84) have been suggested.⁵ The null hypothesis is denoted H₀ with all μ_{G} equal to zero, and the alternative hypothesis is denoted H₁ with at least one non-zero μ_{G}.

An exact calculation of the repository size required to attain desired error rates for a specified genetic model proceeds as follows. First, a value of the pooling fraction ρ or the disease prevalence r is selected. A trial repository size N is specified, with the number of individuals n selected per pool set to the integer part of ρN or rN. Next, the probability P₀(i,j,k) of selecting i individuals with genotype A₁A₁, j individuals with genotype A₁A₂, and k individuals with genotype A₂A₂, with i+j+k equal to n, is tabulated using the multinomial distribution

P₀(i,j,k)=[n!/(i!j!k!)](p²)ⁱ(2p−2p²)^{j}(1−2p−² )^{k}.

The frequency of allele A₁ for this pool composition is (2i+j)/2n. The probability that two pools selected in this manner differ in frequency by at least Δp is calculated as the sum of P₀(i,j,k)P₀(i′j′,k′) for all combinations of i,j,k and i′j′,k′ where

[2(i−i′)+(j−j′)]/2n≧Δp.

Significance at level α is attained by increasing Δp until this sum is less than or equal to α. If not even the maximum value Δp=1 is sufficient for significance at level α, then a larger value of N is selected for the current value of ρ and the calculation begins anew. Otherwise, multinomial probabilities for pool compositions are calculated under H₁ using

P_{U}(i,j,k)=[n!/(i!j!k!)]θ^{U}(A₁A₁)ⁱθ_{U}(A₁A₂)^{j}θ_{U}(A₂A₂)^{k}.

for the upper pool and a similar term P_{L}(i′j′,k′), with θ_{L} replacing θ_{U}, for the lower pool. The probability that the allele frequency difference between the upper and lower pools is at least Δp is obtained as the sum of P_{U}(i,j,k)P_{L}(i′j′,k′) for all compositions i,j,k and i′j′,k′ where [2(i−i′)+(j−j′)]/2n≧Δp. If this probability is greater than or equal to β, the current N is feasible for type I error α and type II error β and a smaller value for N is attempted. This process continues until the smallest feasible N is found.

For the affected/unaffected design, this procedure is followed for each value of r. For the tail pool design, the smallest feasible value for N is calculated as a function of ρ, and the entire design is optimized by searching for the pooling fraction ρ with the smallest feasible N.

When each pool contains a large number of individuals and many copies of each allele, the distribution of allele frequencies for the pool approaches a normal distribution. The difference in allele frequencies between pools, which continues to serve as the test statistic, approaches a normal distribution as well. The pool sizes required to achieve specified error rates are obtained accurately in this case by approximating the multinomial distributions of allele frequencies as normal distributions. Under H₀, the mean of the test statistic is zero and the variance is σ₀²/n=p(1−p)/n, derived by noting that the variance of the frequency difference is twice the variance of the mean for a single pool of n individuals. The allele frequency variance for an individual is p(1−p)/2, and averaging over the n individuals reduces the variance by the factor n.

Under H₁, the expected allele frequency difference Δp is

Δp=p_{U}−p_{L}=Σ_{G}[θ_{U}(G)−θ_{L}(G)]p_{G},

where the genotype-dependent allele frequency p_{G} is 1 for G=A₁A₁, 0.5 for A₁A₂, and 0 for A₂A₂. The variance is σ₁²/n, where σ₁²is obtained from the multinomial distribution,

σ₁²=Σ_{G}[θ_{U}(G)+θ_{L}(G)]p_{G}²−(p_{U}³+p_{L}²).

The repository size N required for type I error a and power 1−β is

n=[z_{α}σ₀−z_{1-β}σ₁]²/Δp².

For tail pools, p is then varied to find the smallest N. The normal approximation underestimates the repository size requirement relative to the exact results from the multinomial distribution. When the sum of the alleles in both pools is at least 60, the difference in repository sizes is no greater than 5%. We chose 60 alleles in both pools as the criterion for switching from the multinomial to the normal calculation. Standard algorithms were employed to perform the root search for X_{U} and X_{L}, the optimization, and the integration over the tail of a normal distribution.

In the regime of typical complex traits, the effect of any single QTL is small, the residual variance σ_{R}² is nearly 1, and analytical results may be obtained by expanding Δp to second order in the effect size μ_{G}. This corresponds loosely to a perturbation theory for probability distributions. The Δp expansion in turn requires a Taylor series expansion for Φ(z),

Φ(z−ε)=Φ(z)−ε(d/dz)Φ(z)+(½)δ²(d/dz)²Φ(z),

truncated at second order. The first derivative is

$$( ⅆ / ⅆ z ) {( 2 π )}^{- 1 / 2} {\int }_{- ∞}^{z} ⅆ {z}^{′} exp ( - {z}^{2} / 2 ) = {( 2 π )}^{- 1 / 2} exp ( - {z}^{2} / 2 ) ≡ y ,$$

where y is the height of the normal distribution at normal deviate z, and the second derivative is

(d/dz)(2π)^{-1/2}exp(−z²/2)=−yz.

Summing these terms,

Φ(z−δ)=Φ(z)−yδ−(½)yzδ².

Substituting this approximation into the expressions for θ(G) using δ=μ_{G}/σ_{R} and z=Φ⁻¹(1−ρ) yields for the tail design

p_{U}=p+(y/ρσ_{R}){Σ_{G}P(G)p_{G}μ_{G}+(}y|z|/2ρσ_{R}²){Σ_{G}P(G)p_{G}μ_{G}²} and

p_{L}=p−(y/ρσ_{R}){Σ_{G}P(G)p_{G}μ_{G}+(}y|z|/2ρσ_{R}²){Σ_{G}P(G)p_{G}μ_{G}²}.

The corresponding expressions for the affected/unaffected pools, with z=Φ⁻¹(1−r), are

p_{U}=p+[y/rσ_{R}]{Σ_{G}P(G)p_{G}μ_{G}+[}y|z|/2rσ_{R}²]{Σ_{G}P(G)p_{G}μ_{G}²} and

The required sums are

Σ_{G}P(G)p_{G}μ_{G}=σ_{A}[p(1−p)/2]^{½}, and

Σ_{G}P(G)p_{G}μ_{G}²=(½)(1−σ_{R}²)−4p²(1−p)²ad+(2p−1)σ_{D}²/2≈σ_{A}²/2.

The approximate value σ_{A}²/2 for the second sum neglects the dominance variance and is exact for purely additive inheritance. It serves to simplify the final equations for Δp. Little error is made in the resulting Δp for two reasons: first, even with dominant or recessive inheritance, the additive variance is often larger than the dominance variance; second, this factor is part of a correction term that is already small.

The results for Δp are

Δp=2^{1/2}yσ₀σ_{A}/ρσ_{R}, tail pools, and

Δp=[1+Φ₋₁(1−r)σ_{A}/2^{3/2}σ₀σ_{R}]yσ₀σ_{A}/2^{1/2}r(1−r)σ_{R}, affected/unaffected pools.

To the same order of approximation, σ₁² may be equated with σ₀², and the number of individuals required per pool is

n=[z_{α}−z_{1-β}]² σ₀²/Δp².

The preceding three equations lead directly to our main results, Eqs. 1 and 2.

The perturbation theory above is valid when the expansion parameters μ_{G}/σ_{R} are small, typically satisfied when σ_{A}²/2p(1−p) is smaller than 1. In this regime, approximate genotype relative risks may be obtained from the Taylor series expansion for θ(G). To lowest order, the relative risk for the heterozygote is 1+(d+a)y/rσ_{R}, and for the A₁A₁ homozygote is 1+2ay/rσ_{R}. For additive inheritance, d=0, and the relative risk is multiplicative with allele dose when ay/rσ_{R} is small.

If individual genotypes are measured for the N individuals in the population, the regression coefficient b₁ in the regression model

X=b₁(p_{G}−p)+ε

is a suitable test statistic. The residual contribution ε to the phenotype has mean zero and is uncorrelated with p_{G}. Under H₀, b₁ has mean zero and variance

Var(b₁|H₀)=N⁻¹Var(X)/Var(p_{G})=1/N[p(1−p)/2].

Under H₁, the expected value and the variance of b₁ are

E(b₁|H₁)=Cov(X,p_{G})/Var(X)=σ_{A}[p(1−p)/2]^{1/2} and

Var(b₁|H₁)=N⁻¹Var(ε)/Var(p_{G})=σ_{R}²/N[p(1−p)/2].

The repository size required for a one-sided test of b₁ with Type I error α and power 1−β is

N=[z_{α}Var(b₁|H₀)^{1/2}−z_{1-β}Var(b₁|H₁)^{1/2}]²/[E(b₁|H₁)]²,

which is presented in simplified form as Eq. 3.

### EXAMPLE 5.1

Two experimental designs are considered using DNA pooled from individuals selected from a pre-existing repository of N samples: affected/unaffected pools, with DNA pooled from n affected and n unaffected individuals; and tail pools, with DNA pooled from the n most extreme individuals at each tail of the phenotype distribution.

For the affected/unaffected design, the expected number of affected individuals is n=rN, and an additional n suitably matched controls are selected from the remainder of the population.

An analytical approximation for the repository size is

N_{aff/unaff}=[z_{α}−z_{1-β}]²[/σ_{R}²/σ_{A}²]·2r(1−r)²/{yᵣ²[1+Φ⁻(1−r)σ_{A}/2^{3/2}σ_{R}p^{1/2}(1−p)^{1/2}]²}, (Eq. 1)

where yᵣ is the height of the standard normal distribution at Φ⁻¹(r) (see Materials and Methods for derivation). Repository size requirements are minimized with a prevalence of 50%,1, much larger than values realistic for complex disorders.

The tail pools are parameterized by the fraction ρ n/N of population N selected for each pool. An analytical approximation for the repository size is

Nₜₐᵢₗ[z_{α}−z_{1-β}]²[σ_{R}²/σ_{A}²]·ρ/2y_{ρ}², (Eq. 2)

where y_{ρ} is the height of the standard normal distribution at Φ⁻¹(ρ) (see Materials and Methods for derivation). The design is optimized by selecting ρ to minimize ρ/2y_{ρ}² and hence N^{tail}. The optimal fraction, 27.03%, is independent of all remaining parameters.

The repository size required to achieve the same error rates using individual genotyping is

N_{indiv}=[z_{α}−z_{1-β}σ^{R]}²/σ_{A}², Eq. 3)

based on a regression model of phenotypic value on allele dose (see Materials and Methods for derivation).

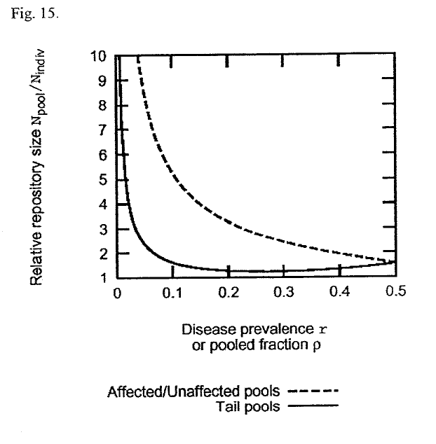

Results of the analytical approximations are shown in FIG. 15 with individual genotyping serving as a reference. The tail design, with ρ=27% of the population selected for each pool, requires a repository only 1.24× larger than required for individual genotyping. It is also robust to variation in ρ near its optimum, as values from 19% to 37% drop the efficiency no more than 5%. In contrast, for 10% disease prevalence, the affected/unaffected design requires a repository 5.3× larger than that required for individual genotyping and is 4× less efficient than the tail design.

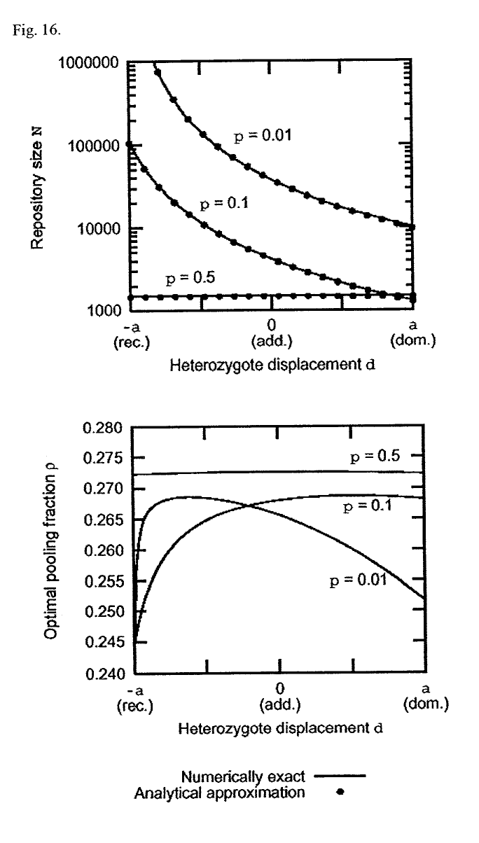

The effect of varying the inheritance mode is shown in FIG. 16 for tail pools. In this example, the type I error is 5×10⁻⁸, the type II error is 0.2, and the displacement a is 0.25 in units of the phenotypic standard deviation. The heterozygote displacement d varies from −a, pure recessive inheritance, to +a, pure dominant inheritance. Results are shown for three frequencies of allele Al: p=0.5, 0.1, and 0.01. Solid lines correspond to exact numerical calculations. In the top panel showing the repository size N, filled circles correspond to analytical approximations, Eq. 1, and are virtually indistinguishable from exact calculations. When p=0.5, A₁ and A₂ have equal frequencies, the additive variance is 0.03125, and the dominance variance is 0 regardless of inheritance mode. Since the population requirements depend primarily on the additive variance, N is independent of the inheritance mode. For allele frequencies below 0.5, the additive variance increases from left to right and the population requirements decreases. The maximum population is required when d equals a/(2p −1), which always falls outside the range depicted. The bottom panel depicts the corresponding values of ρ from the numerical calculations. The optimal pooling fractions fall in a narrow range from 24.5% to 27.5%, close to the analytical approximation of 27.03%.

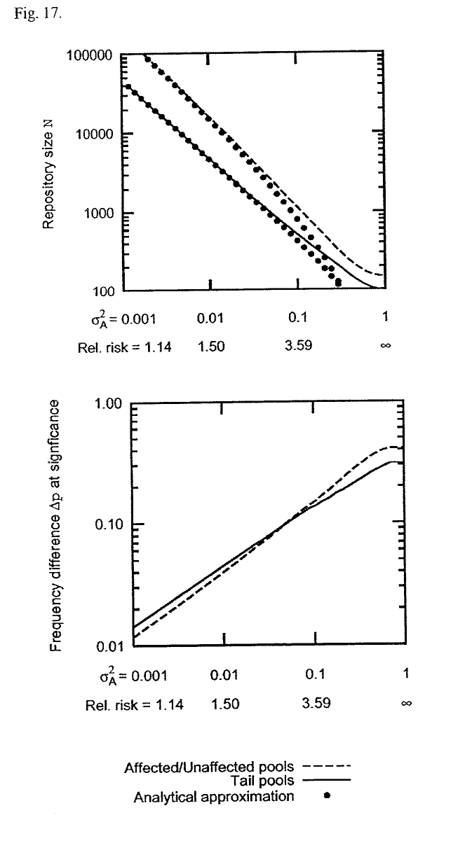

The effect of varying the additive variance directly, or equivalently the genotype relative risk for an allele of known frequency, is shown in FIG. 17. The top panel of FIG. 17 shows that analytical approximations for N from Eqs. 1 and 2 (solid circles) are nearly indistinguishable from the exact numerical results (dashed and solid lines) when the genotype relative risk is below a factor of 2 to 3. Type I and II error rates are 5×10⁻⁸ and 0.2 respectively, and the allele frequency is 0.1. The bottom panel shows the corresponding allele frequency difference that must be measured for a significant finding with a test of pooled DNA. For example, alleles carrying a 1.5× heterozygote relative risk, corresponding to an additive variance of 0.01, have a raw frequency difference of 0.04 at significance: the upper pool has an allele frequency of 0.12 and the lower pool a frequency of 0.08. The population size required to achieve significance is 4700, with 1270 individuals selected per pool.

This analysis assumes that allele frequency measurement error is negligible. Allele frequencies measured by most technologies, including PCR amplification, kinetic PCR, denaturing high performance liquid chromatography, single-strand conformation polymorphism, pyrophosphate sequencing, and mass spectrometry, are typically reported with standard errors in the range of 0.01 to 0.02. Assuming a measurement error of 0.01, the measurement error in the frequency difference is larger by a factor of √2, yielding a anal error of 0.014. Based on the measurement error, the allele frequency difference of 0.04 in the example above corresponds to a z-score of 2.86 and a type I error rate of 0.002.

While this error rate is much larger than the error rate of 5×1 0⁻⁸ required for a whole-genome scan, a practical solution is to employ pooled allele frequency measurements as a pre-screen; candidate associations identified by the pre-screen may then be confirmed by individual genotyping of the entire population, or possibly just the extreme tails. Setting a type I error rate for the pre-screen of 0.01 (z-score of 2.33), corresponding to an allele frequency difference of 0.033, implies a 100× savings over an equivalent study that does not employ a pre-screen.

This experimental limitation sets a threshold for the effect size that may be identified in a pooled DNA pre-screen. The relationship between the expected value of Δp and the parameters of the genetic model for a SNP with purely additive inheritance is

Δp=2.44×[z_{α}/(z_{α}−z_{1-β})]p(1−p)a,

where the initial factor of 2.44 arises from the optimized pooled tail design, z_{α} and z_{1-β} correspond to the type I and II errors that would be obtained neglecting measurement error, and a is the phenotypic displacement as before. For use in a pre-screen with a p-value of 0.01 from measurement error alone, z_{α}=2.33 is reasonable. To retain at least 95% of the true associations, β should be no greater than 0.05, with z_{1-β}=−1.64. These parameters yield Δp equal to 1.43×p(1−p)a, or p(1−p)a=0.023 for the 0.033 frequency difference threshold. For a minor allele frequency of 0.1, this corresponds to a displacement a of 0.26 and an additive variance of 0.012; for allele frequencies of 0.5, the displacement is 0.092 and the additive variance is 0.0042. Thus, the pre-screen retains the power to detect markers with additive variance down to 0.5% to 1.5%, depending on the marker frequency.

### REFERENCES

Abecasis, G R, Cardon, L R, Cookson, W O C (2000) A general test of association for quantitative traits in nuclear families. Am J Hum Genet 66: 279-292.

Alderbom A, Kristofferson A, Hammerling U: Determination of single-nucleotide polymorphisms by real-time pyrophosphate DNA sequencing. Genome Res 2000; 1(0; 1249-1258.

Austin M A, King M C, Bawol R D, Hulley S B, Friedman G D (1987) Risk factors for coronary heart disease in adult female twins. Genetic heritability and shared environmental influences. Am J Epidemiol 125: 308-18.

Barcellos L F, Klitz W, Field L L, Tobias R, Bowcock A M, Wilson R, Nelson M P et al. (1997) Association mapping of disease loci, by use of a pooled DNA genomic screen. Am J Hum Genet 61:734-747.

Beyer W H (ed) (1984) CRC Standard Mathematical Tables, 27^{th} Edition. CRC Press, Boca Raton, Fla.

Buetow K H, Edmonson M, MacDonald R, Clifford R, Yip P, Kelley J, Little D P, Strausberg R, Koester H, Cantor C R, Braun A: High-throughput development and characterization of a genomewide collection of gene-based single nucleotide polymorphism markers by chip-based matrix-assisted laser desorption/ionization time-of-flight mass spectrometry. Proc Nat Acad Sci USA 2001; 98; 581-584.

Cargill M, Altshuler D, Ireland J, Sklar P, Ardlie K, Patil N, Shaw N et al. (1999) Characterization of single-nucleotide polymorphisms in coding regions of human genes. Nat Genet 1999 July;22(3):231-238.

Cardon L R (2000) A Sib-Pair Regression Model of Linkage Disequilibrium for Quantitative Traits. Hum Hered. 50:350-358.

Chandler D. Introduction to Modern Statistical Mechanics. New York: Oxford Univ. Press; 1987

Collins A, Lonjou C, Morton N E (2000) Genetic epidemiology of single-nucleotide polymorphisms. Proc Natl Acad Sci USA 96:15173-15177.

Darvasi A, Soller M (1994) Selective DNA pooling for determination of linkage between a molecular marker and a quantitative trait locus. Genetics 138: 1365-1373.

Falconer, D S, MacKay, T F C (1996) Introduction to quantitative genetics. Addison-Wesley, Boston.

Fulker D W, Chemy S S, Cardon L R (1995) Multipoint interval mapping of quantitative trait loci, using sib pairs. Am J Hum Genet 56:1224-1233.

Frank, L (2000) Storm brews over gene bank of Estonian population. Science 286: 1262.

Germer S, Holland M J, Higuchi R. High-throughput SNP allele-frequency determination in pooled DNA samples by kinetic PCR. Gen Res 2000; 10; 258-266.

Goddard K A, Hopkins P J, Hall J M, Witte J S (2000) Linkage disequilibrium and allele-frequency distributions for 114 single-nucleotide polymorphisms in five populations. Am J Hum Genet 66:216-34.

Gu C, Todorov A, Rao D C (1996) Combining extremely concordant sibpairs with extremely discordant sibpairs provides a cost effective way to linkage analysis of quantitative trait loci. Genet Epidemiol 13:513-533

Heller D A, de Faire U, Pedersen N L, Dahlen G, McCleam G E (1993) Genetic and environmental influences on serum lipid levels in twins. N Engl J Med 328: 1150-6.

Hoogendoorn B, Norton N, Kirov G, Williams N, Hamshere M L, Spurlock G, Austin J, Stephens M K, Buckland P R, Owen M J, O'Donovan M C: Cheap, accurate and rapid allele frequency estimation of single nucleotide polymorphisms by primer extension and DHPLC in DNA pools. Hum Gen 2000; 107; 488-493.

Iselius L, Morton N E, Rao D C (1983) Family resemblance for blood pressure. Hum Hered 33: 277-286.

Kruglyak, L (1999) Prospects for whole-genome linkage disequilibrium mapping of common disease genes. Nature Genetics 22: 139-144.

Kruglyak L, Lander E S (1995) Complete multipoint sib-pair analysis of qualitative and quantitative traits. Am J Hum Genet 57:439-454.

Liu, B -H (1997) Statistical Genomics. CRC Press, Boca Raton.

Mathews J, Walker R L (1970) Mathematical methods of physics, second edition. Benjamin/Cummings, London.

Neale, M C and Cardon, L R (1992). Methodology for Genetic Studies of Twins and Families, NATO ASI Series D, Behavioural and Social Sciences, Vol. 67. Kluwer Academic, Dordrecht.

Nilsson A, Rose J (1999) Sweden takes steps to protect tissue banks. Science 286: 894.

Ott J (1999) Analysis of human genetic linkage. Johns Hopkins Univ Pr, Baltimore.

Perusse L, Rice T, Bouchard C, Vogler G P, Rao D C (1989) Cardiovascular risk factors in a French-Canadian population: resolution of genetic and familial environmental effects on blood pressure by using extensive information on environmental correlates. Am J Hum Genet 45: 240-251.

Press, W H, Teukolsky, S A, Vetterling, W T, and Flannery, B P (1997) Numerical Recipes in C, The Art of Scientific Computing, Second Edition. Cambridge University Press, Cambridge, UK.

Rabinow, P (1999) French DNA: Trouble in Purgatory. University of Chicago Press, Chicago.

Risch N J (2000) Searching for genetic determinants in the new millennium. Nature 405: 847-856.

Risch N J, Merikangas K (1996) The future of genetic studies of complex human diseases. Science 273:1516-1517.

Risch N J, Teng J (1998) The relative power of family-based and case-control designs for linkage disequilibrium studies of complex human diseases I. DNA pooling. Genome Res 8:1273-1288.

Risch N J, Zhang H (1996) Mapping quantitative trait loci with extreme discordant sib pairs: sampling considerations. Am J Hum Genet 58:836-843.

Sasaki T, Tahira T, Suzuki A, Higasa K, Kukita Y, Baba S, Hayashi K: Precise estimation of allele frequencies of single-nucleotide polymorphisms by a quantitative SSCP analysis of pooled DNA. Am J Hum Gen 2001; 68; 214-218.

Sham, P (1997) Statistics in Human Genetics. Arnold.

Shaw S H, Carrasquillo M M, Kashuk C, Puffenberger E G, Chakravarti A: Allele frequency distributions in pooled DNA samples: applications to mapping complex disease genes. Gen Res 1998; 8; 111-123.

Snedecor and Cochran Snedecor G W, Cochran W G. Statistical Methods. 8^{th} Ed. Ames, Iowa: Iowa State University Press; 1989

Verkasalo P K, Kaprio J, Koskenvuo M, Pukkala E (1999) Genetic predisposition, environment and cancer incidence: a nationwide twin study in Finland, 1976-1995. Int J Cancer 83: 743-749.

Watanabe R M, Valle T, Hauser E R, Ghosh S, Enlksson J, Kohtamaki K, Ehnholm C et al. (1999) Familiality of quantitative metabolic traits in Finnish families with non-insulin-dependent diabetes mellitus. Finland-United States Investigation of NIDDM Genetics (FUSION) Study investigators. Hum Hered 49: 159-168.

Wilk J B, Djousse L, Arnett D K, Rich S S, Province M A, Hunt S C, Crapo R O et al. (2000) Evidence for major genes influencing pulmonary function in the NHLBI family heart, study. Genet Epidemiol 19: 81-94.

Zhang H, Risch N (1995) Extreme discordant sib pairs for mapping quantitative trait. loci in humans. Science 268:1584-1589.

Zhang H, Risch N (1996) Mapping quantitative-trait loci in humans by use of extreme concordant sib pairs: selected sampling by parental phenotypes. Am J Hum Genet 59:951-957.

### Tables

TABLE I
Sib-pair genotype probabilities

| Sib Genotype | | |
|---|---|---|
| G₁ | G₂ | P(G₁, G₂) |
| A₁A₁ | A₁A₁ | p₁⁴ + p₁³p₂ + p₁²p₂²/4 |
| A₁A₁ | A₁A₂ | p₁³p₂ + p₁²p₂²/2 |
| A₁A₁ | A₂A₂ | p₁²p₂²/4 |
| A₁A₂ | A₁A₁ | p₁³p₂ + p₁²p₂²/2 |
| A₁A₂ | A₁A₂ | p₁³p₂ + 3p₁²p₂² + p₁p₂³ |
| A₁A₂ | A₂A₂ | p₁²p₂²/2 + p₁p₂³ |
| A₂A₂ | A₁A₁ | p₁²p₂²/4 |
| A₂A₂ | A₁A₂ | p₁²p₂²/2 + p₁p₂³ |
| A₂A₂ | A₂A₂ | p₁²p₂²/4 + p₁p₂³ + p₂⁴ |

TABLE II
Pooling Designs

| Design Family | Indicators | | | |
|---|---|---|---|---|
| Design | I_{U1} | I_{U2} | I_{L1} | I_{L2} |
| Unrelated | | | | |
| Unrelated- | H(X₁ − X_{U}) | — | H(X_{L} − X₁) | — |
| Random | | | | |
| Unrelated- | H(X₁ − X_{U}) × | H(X₂ − X_{U}) × | H(X_{L} − X₁) × | H(X_{L} − X₂) × |
| Extreme | H(\|X₁\| − \|X₂\|) | H(\|X₂\| − \|X₁\|) | H(\|X₁\| − \|X₂\|) | H(\|X₂\| − \|X₁\|) |
| Sib-Together | | | | |
| Concordant | H(X₁ − X_{U}) × | H(X₁ − X_{U}) × | H(X_{L} − X₁) × | H(X_{L} − X₁) × |
| | H(X₂ − X_{U}) | H(X₂ − X_{U}) | H(X_{L} − X₂) | H(X_{L} − X₂) |
| Pair-mean | H(X₊ − X_{U}) | H(X₊ − X_{U}) | H(X_{L} − X₊) | H(X_{L} − X₊) |
| Sib-Apart | | | | |
| Discordant | H(X₁ − X_{U}) × | H(X_{L} − X₁) × | H(X_{L} − X₁) × | H(X₁ − X_{U}) × |
| | H(X_{L} − X₂) | H(X₂ − X_{U}) | H(X₂ − X_{U}) | H(X_{L} − X₂) |
| Pair-difference | H(\|X₋\| − X_{U}) × | H(\|X₋\| − X_{U}) × | H(\|X₋\| − X_{U}) × | H(\|X₋\| − X_{U}) × |
| | H(X₁ − X₂) | H(X₂ − X₁) | H(X₂ − X₁) | H(X₁ − X₂) |

TABLE III
Sib-pair genotype probabilities

| Sib-Pair | | |
|---|---|---|
| Genotype | | |
| G₁ | G₂ | P(G₁, G₂) |
| A₁A₁ | A₁A₁ | p⁴ + p³(1 − p) + p²(1 − p)²/4 |
| A₁A₁ | A₁A₂ | p³(1 − p) + p²(1 − p)²/2 |
| A₁A₁ | A₂A₂ | p²(1 − p)²/4 |
| A₁A₂ | A₁A₁ | p³(1 − p) + p²(1 − p)²/2 |
| A₁A₂ | A₁A₂ | p³(1 − p) + 3p²(1 − p)² + p(1 − p)³ |
| A₁A₂ | A₂A₂ | p²(1 − p)²/2 + p(1 − p)³ |
| A₂A₂ | A₁A₁ | p²(1 − p)²/4 |
| A₂A₂ | A₁A₂ | p²(1 − p)²/2 + p(1 − p)³ |
| A₂A₂ | A₂A₂ | p²(1 − p)²/4 + p(1 − p)³ + (1 − p)⁴ |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting an association in a population of individuals between a genetic locus and a quantitative phenotype, wherein two or more alleles occur at the locus, and wherein the phenotype is expressed using a numerical phenotypic value whose range falls within a first numerical limit and a second numerical limit, the method comprising the steps of
a) obtaining the phenotypic value for each individual in the population;
b) selecting a first subpopulation of individuals having phenotypic values that are higher than a predetermined lower limit and pooling DNA from the individuals in the first subpopulation to provide an upper pool;
c) selecting a second subpopulation of individuals having phenotypic values that are lower than a predetermined upper limit and pooling DNA from the individuals in the second subpopulation to provide a lower pool;
d) for one or more genetic loci, measuring the difference in frequency of occurrence of a specified allele between the upper pool and the lower pool; and
e) determining that an association exists if the allele frequency difference between the pools is larger than a predetermined value.

2. The method described in claim 1 wherein the lower limit and the upper limit are chosen such that, for a specified false-positive rate, the frequency of occurrence of false-negative errors is minimized.

3. The method described in claim 1 wherein the population comprises unrelated individuals.

4. The method described in claim 1 wherein the population comprises related individuals.

5. The method described in claim 3 wherein the predetermined lower limit is set so that the upper pool includes the highest 35% of the population and the predetermined upper limit is set so that the lower pool includes the lowest 35% of the population.

6. The method described in claim 3 wherein the predetermined lower limit is set so that the upper pool includes the highest 30% of the population and the predetermined upper limit is set so that the lower pool includes the lowest 30% of the population.

7. The method described in claim 3 wherein the predetermined lower limit is set so that the upper pool includes the highest 27% of the population and the predetermined upper limit is set so that the lower pool includes the lowest 27% of the population.

8. The method described in claim 2 wherein the individuals in the population are sibling pairs and each pair is ranked according to the phenotypic values of the siblings in each pair, and either (i) both members of the sibling pair are selected for the upper pool; (ii) both members of the sibling pair are selected for the lower pool; or (iii) neither member of the sibling pair is selected.

9. The method described in claim 8 wherein each sibling pair is ranked according to a mean value of the phenotypic values of the siblings in each pair, and wherein both members of the sibling pair are in the same pool.

10. The method described in claim 8 wherein the phenotypic values of the siblings in each pair are both above a predetermined lower limit or both below a predetermined upper limit.

11. The method described in claim 8 wherein the predetermined lower limit is set so that the upper pool includes the pairs with the highest 10% of the mean values in the population and the predetermined upper limit is set so that the lower pool includes the lowest 10% of the mean values in the population.

12. The method described in claim 8 wherein the predetermined lower limit is set so that the upper pool includes the pairs with the highest 15% of the mean values in the population and the predetermined upper limit is set so that the lower pool includes the lowest 15% of the mean values in the population.

13. The method described in claim 8 wherein the predetermined lower limit is set so that the upper pool includes the pairs with the highest 20% of the mean values in the population and the predetermined upper limit is set so that the lower pool includes the lowest 20% of the mean values in the population.

14. The method described in claim 8 wherein the predetermined lower limit is set so that the upper pool includes the pairs with the highest 25% of the mean values in the population and the predetermined upper limit is set so that the lower pool includes the lowest 25% of the mean values in the population.

15. The method described in claim 8 wherein the predetermined lower limit is set so that the upper pool includes the pairs with the highest 27% of the mean values in the population and the predetermined upper limit is set so that the lower pool includes the lowest 27% of the mean values in the population.

16. The method described in claim 2 wherein all individuals in the population are members of sibling pairs, and either (i) one member of a sibling pair is selected for the upper pool and the second member of the sibling pair is selected for the lower pool; or (ii) neither member of a sibling pair is selected.

17. The method described in claim 17 wherein the sibling pairs are ranked by the absolute magnitude of the difference in phenotypic value for the siblings within each pair, the percent of pairs with the greatest difference are identified, and the siblings in each pair are distributed such that the sibling with the high phenotypic value is selected for the upper pool and the sibling with the low phenotypic value is selected for the lower pool.

18. The method described in claim 17 wherein the phenotypic value of one member of the sibling pair is above a predetermined lower limit and the phenotypic value of the second member of the sibling pair is below a predetermined upper limit.

19. The method described in claim 17 wherein the percent of pairs is 80% and the distribution provides 10% of the population in each pool.

20. The method described in claim 17 wherein the percent of pairs is 70% and the distribution provides 15% of the population in each pool.

21. The method described in claim 17 wherein the percent of pairs is 60% and the distribution provides 20% of the population in each pool.

22. The method described in claim 17 wherein the percent of pairs is 50% and the distribution provides 25% of the population in each pool.

23. The method described in claim 17 wherein the percent of pairs is 54% and the distribution provides 27% of the population in each pool.

24. The method described in claim 2 wherein the individuals in the population are sibling pairs and the results obtained by performing the methods described in claims 7 and 15 are combined.

25. The method described in claim 3 wherein the population of unrelated individuals are provided by a process comprising the steps of:
a) providing a population of sibling pairs; and
b) selecting only one member of a sibling pair to be included in the population of unrelated individuals.

26. The method described in claim 25 further comprising the steps of:
a) calculating the overall mean of the phenotypic values in the population;
b) subtracting the mean from each phenotypic value;
c) ranking each sibling pair according to the result of the calculation conducted according to
(pair-mean)²/(variance of pair-mean)+(pair-difference)²/(variance of pair difference) to provide the Mahalanobis rank;
d) identifying a more extreme sibling from each sibling pair as the member of the pair having a greater magnitude of the phenotypic value; and
e) from sibling pairs having extreme Mahalanobis ranks constructing pools using the sibling of the pair having the more extreme phenotypic value.

27. The method described in claim 25, further comprising the steps of:
a) calculating the overall mean of the phenotypic values in the population; and
b) selecting that member of each sibling pair having a phenotypic value such that the absolute value of the difference between the individual's phenotypic value and the overall mean is greater than the difference for the other individual in the pair,
thereby providing a population of unrelated individuals.

28. The method described in claim 25 further comprising the steps of:
a) rank ordering the members of the population of sibling pairs to generate a list wherein the rank order of each member of a sibling pair is obtained as the smaller of:
i) the distance from the first member on the list and
ii) the distance from the last member on the list; and
b) selecting that member of each sibling pair having a lower ranking; thereby providing a population of unrelated individuals.

29. The method described in claim 25 further comprising the steps of:
a) rank ordering the members of the population of sibling pairs to generate a list wherein the rank order of each member of a sibling pair is obtained as the distance from the phenotype mean; and
b) selecting that member of each sibling pair having a lower ranking; thereby providing a population of unrelated individuals.

30. The method described in claim 1 wherein the population includes individuals who may be classified into classes.

31. The method described in claim 30 wherein the classes are based on an age group, gender, race or ethnic origin.

32. The method described in claim 31 wherein all the members of a class are included in the pools.

33. The method described in claim 1 for determining the genetic basis of disease predisposition.

34. The method described in claim 33, wherein the genetic locus which is analyzed for determining the genetic basis of disease predisposition contains a single nucleotide polymorphism.
